(19) United States
(12) Patent Application Publication
(10) Pub. No.: US 2013/0281442 A1
(43) Pub. Date: Oct. 24, 2013

(54) Compounds for Treatment of Bovine Mastitis

(71) Applicant: Barry Hafkin, Austin, TX (US)

(72) Inventor: Barry Hafkin, Austin, TX (US)

(21) Appl. No.: 13/703,406

(22) PCT Filed: Jun. 13, 2011
(86) PCT No.: PCT/US11/40187

§ 371 Date: Jun. 18, 2013

Related U.S. Application Data
(60) Provisional application No. 61/353,918, filed on Jun. 11, 2010.

(51) Int. Cl. C07D 471/04

(52) U.S. Cl. CPC C07D 471/04
USPC 514/221; 514/300

(57) ABSTRACT

Described herein are methods of treating mastitis in female mammals, e.g., cows, wherein the methods may include administering to mammals in need thereof compounds disclosed herein.

### CROSS REFERENCE TO RELATED APPLICATIONS

This application claims priority to U.S. Ser. No. 61/353,918, entitled, “Compounds for Treatment of Bovine Mastitis,” filed Jun. 11, 2010, which is hereby incorporated by reference in its entirety.

### BACKGROUND

The spread of bacterial infection in connection with cow teats during the milking process results in the spread of the infectious mammary disease known as mastitis. Bovine mastitis is an inflammation of the udder. The characteristic features of inflammation are swelling, heat, redness, pain, and disturbed function. This condition, which is almost exclusively initiated by pathogenic microorganisms that have entered the teat canal after the milking process, occludes milk flow and production, decreases milk value, and may permanently impair an animal's ability to produce milk.

More than 80 species of microorganisms have been identified as causal agents for bovine mastitis, although approximately 95% of such mastitis is believed to be caused by four pathogens: Staphylococcus aureus, Streptococcus agalactiae, Streptococcus dysagalactiae, and Streptococcus uberis. Mastitis-causing pathogens typically fall into two categories, namely, contagious and environmental. Contagious bacteria, such as streptococcus agalactiae and staphylococcus aureus, primarily colonize host tissue sites such as mammary glands, teat canals, and teat skin lesions; and are spread from one infected cow to another during the milking process. Environmental bacteria, often streptococci, enterococci, and coliform organisms, are commonly present within the cow's surroundings from sources such as cow feces, soil, plant material, bedding, or water; and infect by casual opportunistic contact with an animal.

Examples of potential bacterial targets are those enzymes involved in fatty acid biosynthesis. While the overall pathway of saturated fatty acid biosynthesis is similar in all organisms, the fatty acid synthase (FAS) systems vary considerably with respect to their structural organization. Vertebrates and yeast possess a FAS in which all the enzymatic activities are encoded on one or two polypeptide chains, respectively, and the acyl carrier protein (ACP) is an integral part of the complex. In contrast, in bacterial FAS, each of the reactions is catalyzed by a distinct, mono-functional enzyme and the ACP is a discrete protein. Therefore, there is considerable potential for the selective inhibition of the bacterial system by antibacterial agents. FabI is a major biosynthetic enzyme and is a key regulatory point in the overall synthetic pathway of bacterial fatty acid biosynthesis, and may an ideal target for antibacterial intervention.

Importantly, it has now been discovered that certain compounds may be useful for the treatment of bacterial infections in mammals such as cows afflicted by bovine mastitis.

### SUMMARY

Described herein are methods of treating mastitis in female mammals, e.g., cows, wherein the methods may include administering to mammals in need thereof compounds disclosed herein.

In one aspect, a method of treating mastitis in a female mammal in need thereof is provided. The method comprises administering to the female mammal having or at risk of having mastitis an effective amount of a compound selected from the group consisting of (E)-N-methyl-N-((2-methyl-1H-indol-3-yl)methyl)-3-(7-oxo-5,6,7,8-tetrahydro-1,8-naphthyridin-3-yl)acrylamide; (E)-N-methyl-N-(3-methylbenzofuran-2-yl)methyl)-3-(2,3,4,5-tetrahydro-1H-pyrido[2,3-e][1,4]diazepin-7-yl)acrylamide; or (E)-N-methyl-N-(3-methylbenzofuran-2-ylmethyl)-3-(7-oxo-5,6,7,8-tetrahydro-1,8-naphthyridin-3-yl)acrylamide or a pharmaceutically acceptable salt or ester thereof.

In some embodiments, the female mammal is a milk producing mammal.

In some embodiments, the female mammal is a cow, horse, human, goat, sheep, buffalo, or camel.

In another aspect, a method of treating bovine mastitis in a cow in need thereof is provided. The method comprises administering to said cow an effective amount of a composition comprising (E)-N-methyl-N-((2-methyl-1H-indol-3-yl)methyl)-3-(7-oxo-5,6,7,8-tetrahydro-1,8-naphthyridin-3-yl)acrylamide or a pharmaceutically acceptable salt or ester thereof.

In yet another aspect, a method of treating bovine mastitis in a cow in need thereof is provided. The method comprises administering to said cow an effective amount of a composition comprising (E)-N-methyl-N-((3-methylbenzofuran-2-yl)methyl)-3-(2,3,4,5-tetrahydro-1H-pyrido[2,3-e][1,4]diazepin-7-yl)acrylamide or a pharmaceutically acceptable salt or ester thereof.

In some embodiments, the mastitis is caused by a bacterial infection.

In some embodiments, the bacterial infection is caused by one or more strains of Staphylococcus aureus.

In some embodiments, the bacterial infection is caused by one or more strains of Staphylcoccus Oxford, Staphylococcus aureus WCUH29, Streptococcus pneumoniae ERY2, Streptococcus pneumoniae 1629, Streptococcus pneumoniae N 1387, Enterococcus faecalis I, Enterococcus faecalis 7, Haemophilus influenzae Q1, Haemophilus influenzae NEMC1, Moraxella Catarrhalis 1502, Escherichia coli 7623 AcrABEFD+, Escherichia coli 120 AcrAB-, Escherichia coli MG1655, or Escherichia coli MG1658.

In some embodiments, the bacterial infection is caused by one or more strains of Staphylococcus auereues, Streptococcus dysgalactiae, Streptococcus equinus, Streptococcus agalactiae, Staphylococcus hyicus, Staphylococcus simulans, Staphylococcus epidermidis, Staphylococcus chromogenes or Staphylococcus xylosus.

In some embodiments, the bacterial infection is caused by one or more strains of Pseudomonas aeruginosa, Corynebacterium pyogenes, Mycoplasma Bovis, Serratia, Candida, E. coli, Klebsiella or Enterobacter.

In some embodiments, the S. aureus is methicillin-resistant Staphylococcus aureus.

In some embodiments, the compound is administered to the udder of the cow.

In some embodiments, the compound is administered orally, rectally, vaginally, subcutenously, or intravenously.

### DETAILED DESCRIPTION

### Definitions

For convenience, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used to mean “including but not limited to”. “Including” and “including but not limited to” are used interchangeably.

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Antibiotic agents and FabI/FabK inhibitors are examples of therapeutic agents.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions may be administered in a sufficient amount to produce a at a reasonable benefit/risk ratio applicable to such treatment.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

The term “aliphatic” is art-recognized and refers to a linear, branched, cyclic alkane, alkene, or alkyne. In certain embodiments, aliphatic groups are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

Moreover, the term “alkyl” (or “lower alkyl”) includes “substituted alkyls”, which refers to alkyl moieties having substituents replacing a hydrogen on one or more carbons of the hydrocarbon backbone. Such substituents may include, for example, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CN and the like. Exemplary substituted alkyls are described below. Cycloalkyls may be further substituted with alkyls, alkenyls, alkoxys, alkylthios, aminoalkyls, carbonyl-substituted alkyls, —CN, and the like, e.g., C₃₋₇cycloalkyl refers to an optionally substituted carbocyclic system of three to seven carbon atoms, which may contain up to two unsaturated carbon-carbon bonds. Typical of C₃₋₇cycloalkyl are cyclopropyl, cyclobutyl, cyclopentyl, cyclopentenyl, cyclohexyl, cyclohexenyl and cycloheptyl. Any combination of up to three substituents, such as those defined above for alkyl, on the cycloalkyl ring that is available by conventional chemical synthesis and is stable, is contemplated.

For example, C₁₋₄ alkyl as applied herein means an optionally substituted alkyl group of 1 to 4 carbon atoms, and includes methyl, ethyl, n-propyl, isopropyl, n-butyl, isobutyl and t-butyl. C₁₋₆alkyl additionally includes pentyl, n-pentyl, isopentyl, neopentyl and hexyl and the simple aliphatic isomers thereof. C₀₋₄ alkyl and C₀₋₆ alkyl additionally indicates that no alkyl group need be present (e.g., that a covalent bond is present). Any C₁₋₄ alkyl or C₁₋₆ alkyl may be optionally substituted with the group R^{x}, which may be on any carbon atom that results in a stable structure and is available by conventional synthetic techniques. Suitable groups for R^{x} are C₁₋₄ alkyl, OR′, SR′, CN, N(R′)₂, CH₂N(R′)₂, NO₂, CF₃, CO₂R′ CON(R′)₂, COR′, —NR′C(O)R′, F, Cl, Br, I, or —S(O)ᵣCF₃,wherein R′ and r are as defined for formula (I) compounds.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls. In some embodiments, Ar, or aryl, as applied herein, means phenyl or naphthyl, or phenyl or naphthyl substituted by one to three substituents, such as those defined above for alkyl, or substituted by methylenedioxy.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like. Het, or heterocycle, indicates an optionally substituted five or six membered monocyclic ring, or a nine or ten-membered bicyclic ring containing one to three heteroatoms chosen from the group of nitrogen, oxygen and sulfur, which are stable and available by conventional chemical synthesis. Illustrative heterocycles are benzofuryl, benzimidazolyl, benzopyranyl, benzothienyl, furyl, imidazolyl, indolinyl, morpholinyl, piperidinyl, piperazinyl, pyrrolyl, pyrrolidinyl, tetrahydropyridinyl, pyridinyl, thiazolyl, thienyl, quinolinyl, isoquinolinyl, and tetra- and perhydro- quinolinyl and isoquinolinyl. Any accessible combination of up to three substituents on the Het ring, such as those defined above for alkyl, that are available by chemical synthesis and are stable are within the scope of this invention.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂^{—}. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

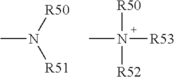

wherein R50, R51 and R52 each independently represent a hydrogen, an alkyl, an alkenyl, —(CH₂)ₘ-R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ-R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

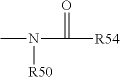

wherein R50 is as defined above, and R54 represents a hydrogen, an alkyl, an alkenyl or —(CH₂)ₘ-R61, where m and R61 are as defined above.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O.

Certain compounds contained in compositions may exist in particular geometric or stereoisomeric forms. In addition, polymers may also be optically active. Contemplated herein are all such compounds, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are intended to be included in this invention.

If, for instance, a particular enantiomer of a compound is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds that may be substituted or unsubstituted.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

The term “hydroxyl-protecting group” is art-recognized and refers to those groups intended to protect a hydrozyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Non-limiting examples of amino-blocking groups include benzyl (—CH₂C₆H₅), acyl[C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g., lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

A “patient,” “subject” or “host” to be treated by the subject method may mean either a human or non-human animal.

The term “mammal” is known in the art, and exemplary mammals include humans, primates, bovines, porcines, canines, felines, and rodents (e.g., mice and rats).

The term “bioavailable” is art-recognized and refers to a form of the subject invention that allows for it, or a portion of the amount administered, to be absorbed by, incorporated to, or otherwise physiologically available to a subject or patient to whom it is administered.

The term “pharmaceutically-acceptable salts” is art-recognized and refers to the relatively non-toxic, inorganic and organic acid addition salts of compounds, including, for example, those contained in the compositions.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Provided herein are methods of treating a female mammal, e.g. a milk-producing mammal such as a cow, horse, human, goat, sheep, buffalo, or camel.

Mastitis can be caused by bacteria; for example, bovine mastitis may be caused primarily by bacteria and/or may be caused by yeasts and molds. In some cases the causes of bovine mastitis are unknown and could be due to physical trauma or weather extremes. Although bovine mastitis can be caused by many different bacterial species, the most common are the Staphylococcus and Streptococcus species.

The most common staphylococci and streptococci causing bovine mastitis include Staphylococcus auereues, Streptococcus dysgalactiae, Streptococcus equinus, Streptococcus agalactiae, Staphylococcus hyicus, Staphylococcus simulans, Staphylococcus epidermidis, Staphylococcus chromogenes and Staphylococcus xylosus. Other staphylococci and streptococci known to cause bovine mastitis include Staphylcoccus Oxford, Staphylococcus aureus WCUH29, Streptococcus pneumoniae ERY2, Streptococcus pneumoniae 1629, Streptococcus pneumoniae N 1387, Enterococcus faecalis I, Enterococcus faecalis 7, Haemophilus influenzae Q1, Haemophilus influenzae NEMC1, Moraxella Catarrhalis 1502, Escherichia coli 7623 AcrABEFD+, Escherichia coli 120 AcrAB-, Escherichia coli MG1655, or Escherichia coli MG1658. In some embodiments, the organism may be methicillin-resistant Staphylococcus aureus.

In some embodiments, bovine mastitis may also be caused by gram-negative bacteria, or by organisms such as Pseudomonas aeruginosa, Brucella melitensis, Corynebacterium bovis, various species of Mycoplasma, Escherichia coli, Klebsiella pneumoniae, Klebsiella oxytoca, Enterobacter aerogenes, various species of Pasteurella, Arcanobacterium pyogenes, various species of Proteus, Prototheca zopfii (e.g., achlorophyllic algae), and Prototheca wickerhamii (e.g., achlorophyllic algae).

For example, provided herein are methods for treatment of mastitis, such as bovine mastitis, including administering to a mammal (e.g. a cow) in need thereof a pharmaceutically effective amount of a compound represented by Formula (I):

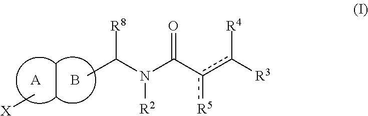

wherein:

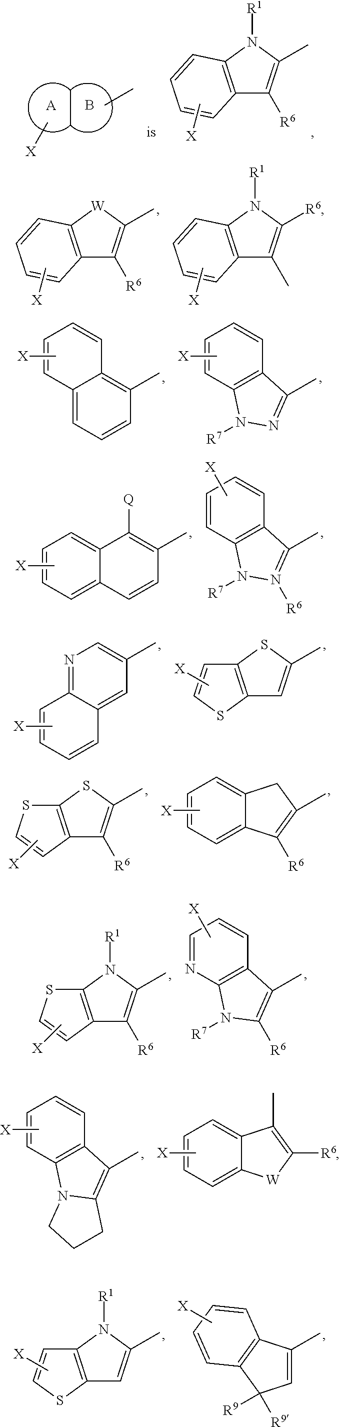

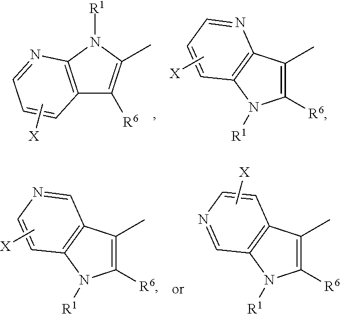

R¹ is H, C₁₋₄alkyl, —C₀₋₆alkyl-Ar, —(CH₂)₁₋₃N(R′)₂, or —(CH₂)₁₋₃OR′;
R² is H, C₁₋₄alkyl or C₃₋₆cycloalkyl;
R³ is

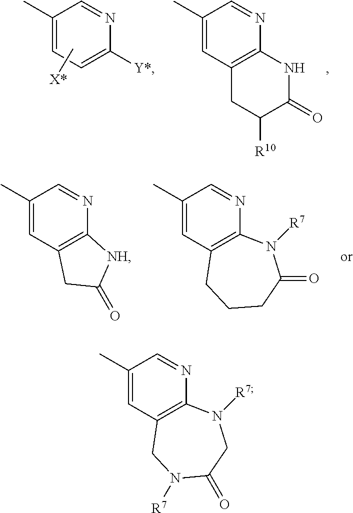

R⁴ is H or C₁₋₄alkyl;
[symbol] indicates that one of the two designated bonds is a double bond and the other is a single bond;
R⁵ is CH₂ when the bond to which it is attached is a double bond; or R⁵ is H or C₁₋₄alkyl when the bond to which it is attached is a single bond;
R⁶ is H or C₁₋₄alkyl;
each R⁷ independently is H, C₁₋₆alkyl, —C₀₋₆alkyl-Ar, —(CH₂)₁₋₃N(R′)₂, or —(CH₂)₁₋₃OR′;
R⁸ is H or C₁₋₄alkyl;
R9 and R⁹′ independently are H or C₁₋₄alkyl;
R¹⁰ is C₁₋₄alkyl, N(R′)₂, NHC(O)R′, NHCH₂C(O)R′ or NHC(O)CH═CHR′;
Y* is N(R′)₂, NHC(O)R′, NHCH₂C(O)R′ or NHC(O)CH═CHR′;
each X independently is H, C₁₋₄alkyl, CH₂OH, OR′, SR′, CN, N(R′)₂, CH₂N(R′)₂, NO₂, CF₃, CO₂R′, CON(R′)₂, COR′, NR′C(O)R′, F, Cl, Br, I or —S(O)ᵣCF₃;
X* is —(CH₂)₁₋₃C(O)N(R′)—(CH₂)₁₋₃-Ar or —(CH₂)₁₋₃C(O)N(R′)—(CH₂)₁₋₃-Het;
W is S or O;
Q is H or C₁₋₄ alkyl;
each R′ independently is H, C₁₋₆alkyl, —C₀₋₆alkyl-Ar or —Co₀₋₆alkyl-Het; and
r is 0, 1 or 2;
or a pharmaceutically acceptable salt thereof.

In another aspect, other compounds contemplated for use in treating mastitis are provided that include compounds of Formula (IV).

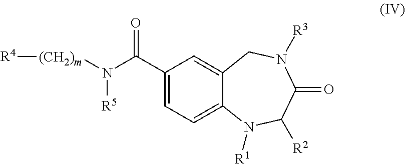

wherein:
R¹ is H, C₁₋₆ alkyl or Ar C₀₋₆ alkyl;
R² is H, C₁₋₆ alkyl, Ar-C₀₋₆ alkyl, HO—(CH₂)n- or R′OC(O)-(CH₂)n-;
R³ is A-C₂₋₄alkyl, A-C₂₋₄alkenyl, A-C₂₋₄ alkynyl, A-C₃₋₄oxoalkenyl, A-C₃₋₄oxoalkynyl, A-C₁₋₄aminoalkyl, A-C₃₋₄aminoalkenyl, A-C₃₋₄aminoalkynyl, optionally substituted by any accessible combination of one or more of R¹⁰ or R⁷;

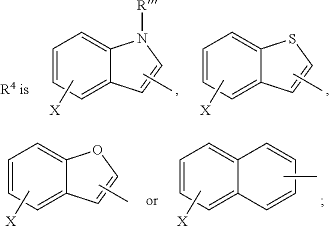

R⁵ is H, C₁₋₆alkyl, Ar-C₀₋₆alkyl or C₃₋₆cycloalkyl-C₀₋₆alkyl;
A is H, C₃₋₆cycloalkyl, Het or Ar;
R⁷ is —COR⁸, —COCR′₂R⁹, —C(S)R⁸, —S(O)ₖOR′, —S(O)ₖNR′R″, —PO(OR′), —PO(OR′)₂, —B(OR′)₂, —NO₂, or tetrazolyl;
R⁸ is —OR′, —NR′R″, —NR′SO₂R′, —NR′OR′, or —OCR′₂CO(O)R′;
R⁹ is OR′, —CN, —S(O)ᵣR′, —S(O)ₖNR′₂, —C(O)R′, C(O)NR′₂, or —CO₂R′;
R¹⁰ is H, halo, —OR¹¹, —CN, —NR′R¹¹, —NO₂, —CF₃, CF₃S(O)r-, —CO₂R′, —CONR′₂, A—C₀₋₆alkyl-, A—C₁₋₆oxoalkyl-, A—C₂₋₆alkenyl-, A—C₂₋₆alkynyl-, A—C₀₋₆alkyloxy-, A—C₀₋₆alkylamino- or A—C₀₋₆alkyl S(O)ᵣ₋;
R¹¹ is R′, —C(O)R′, —C(O)NR′₂, —C(O)OR′, —S(O)ₖR′, or —S(O)ₖNR′₂;
R′ is H, C₁₋₆alkyl, Ar—C₀₋₆alkyl or C₃₋₆cycloalkyl-C₀₋₆alkyl;
R″ is R′, —C(O)R′ or —C(O)OR′;
R″' is H, C₁₋₆alkyl, Ar—C₀₋₆alkyl, HO—(CH₂)₂-, R′C(O)-, (R′)₂NC(O)CH₂- or R′S(O)₂-;
X is H, C₁₋₄alkyl, OR′, SR′, C₁₋₄alkylsulfonyl, C₁₋₄alkylsulfoxyl, —CN, N(R′)₂, CH₂N(R′)₂, —NO₂, —CF₃, —CO₂R′, —CON(R′)₂, —COR′, —NR′C(O)R′, F, Cl, Br, I, or CF₃S(O)ᵣ₋;
k is 1 or 2;
m is 1, 2 or 3;
n is 1 to 6; and
r is 0, 1 or 2;
or a pharmaceutically acceptable salt thereof.

With respect to formula (IV), in some embodiments, R⁴ is:

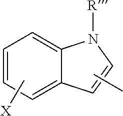

in which R″′ is H, C₁₋₄alkyl or Ar—C₀₋₄alkyl and X is H, C₁₋₄alkyl, OR′, SR′, —CN, —CF₃, —CO₂R′, F, Cl, Br or I.

For example, R³ is H, C₁₋₆alkyl, Ar—C₀₋₆alkyl, Het—C₀₋₆alkyl, C₃₋₆cycloalkyl-C₀₋₆alkyl, —CH₂CF₃, —(CH₂)₁₋₂C(O)OR′, or —(CH₂)₂OR′, wherein R′ is H or C₁₋₄alkyl. In some embodiments, R³ is H, C₁₋₄alkyl or Ph—C₀₋₄alkyl; R¹ may be H and m is 1; R⁵ is H or C₁₋₄alkyl and/or R² is H, C₁₋₄alkyl, Ph—C₀₋₄alkyl, HO-(CH₂)₁₋₂- or R′OC(O)-(CH₂)₁₋₂-, wherein R′ is H or C₁₋₄alkyl.

In another aspect, methods for treatment of mastitis, such as bovine mastitis, including administering to a mammal (e.g. a cow) in need thereof a pharmaceutically effective amount of compounds of formula (VIII) are provided:

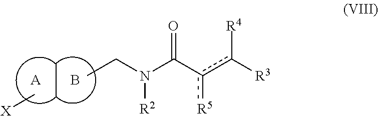

wherein:

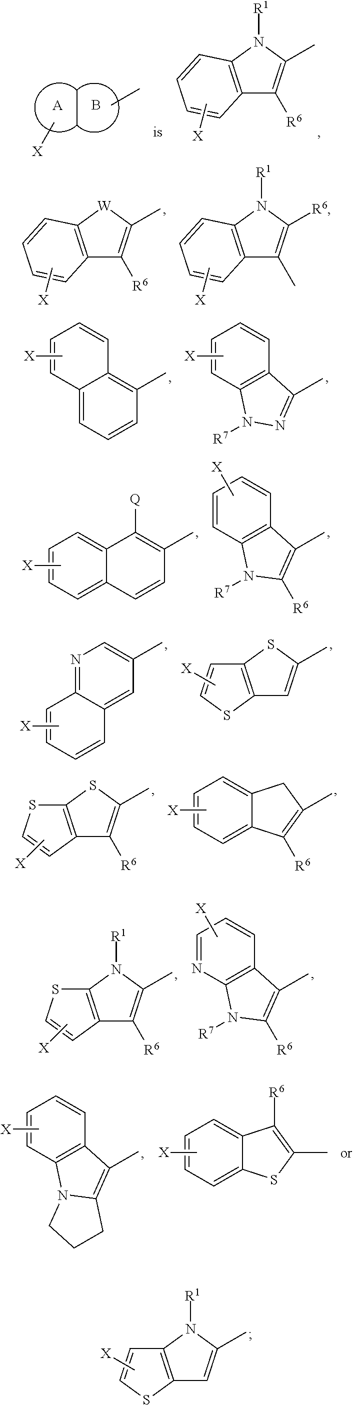

R¹ is H or C₁₋₄alkyl;
R² is H, C₁₋₄alkyl or C₃₋₆cycloalkyl;
R³ is

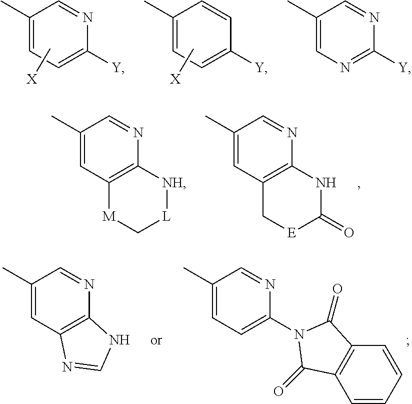

R⁴ is H or C₁₋₄alkyl; [symbol]
   indicates that one of the two designated bonds is a double bond and the other is a single bond;
R⁵ is CH₂ when the bond to which it is attached is a double bond; or R⁵ is H or C₁₋₄alkyl when the bond to which it is attached is a single bond;
R⁶ is H or C₁₋₄alkyl;

R⁷ is H, C₁₋₆alkyl or —C₀₋₆alkyl-Ar;
Y is H, C₁₋₄alkyl, N(R′)₂, NHC(O)R′, NHCH₂C(O)R′ or NHC(O)CH═CHR′;
each X independently is H, C₁₋₄alkyl, CH₂OH, OR, SR, CN, N(R′)₂, CH₂N(R′)₂, NO₂, CF₃, CO₂R′, CON(R′)₂, COR′, NR′C(O)R′, F, Cl, Br, I or —S(O)ᵣCF₃;
W is S or O;
Q is H or C₁₄alkyl;
M is CH₂ or O;
L is CH₂ or C(O);
E is O or NR′;
each R′ independently is H, C₁₋₆alkyl or —Co₀₋₆alkyl-Ar; and
r is 0, 1 or 2;
or a pharmaceutically acceptable salt thereof.

With respect to formula (VIII), the compositions may include compounds of formula (Ia):

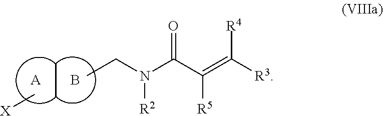

in which R², R³, R⁴, R⁵ and X are as defined for formula (VIII) compounds.

With respect to formula (VIII), the compositions may include compounds of formula (IX):

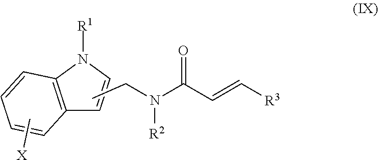

in which R¹, R², R³ and X are as defined for formula (I) compounds.

With respect to formula (IX), the compositions may include compounds of formula (IXa):

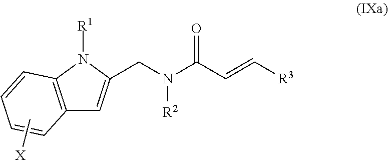

in which R¹, R², R³ and X are as defined for formula (IX) compounds.

In particular, with respect to formula (IX), the compositions may include compounds of formula (IXb):

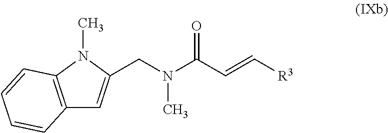

in which R³ is as defined for formula (VIII) compounds.

For example, respect to formula (VIII), R³ may be represented by:

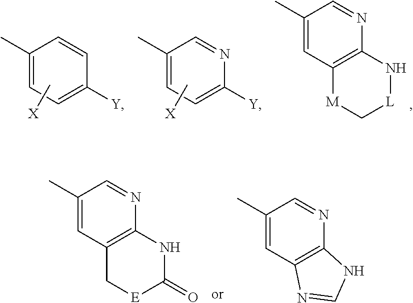

in which X, Y, M, L and E are as defined for formula (VIII) compounds.

In another aspect, a method of treating mastitis (e.g. bovine mastitis) by administering to a mammal (e.g. a cow) in need thereof a pharmaceutically effective amount of a compound including those depicted by formula (XIV):

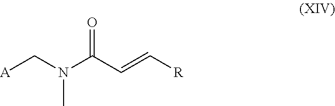

wherein, independently for each occurrence, A is a monocyclic ring of 4-7 atoms containing 0-2 heteroatoms, a bicyclic ring of 8-12 atoms containing 0-4 heteroatoms or a tricyclic ring of 8-12 atoms containing 0-6 heteroatoms wherein the rings are independently aliphatic, aromatic, heteroaryl or heterocyclic in nature, the heteroatoms are selected from N, S or O and the rings are optionally substituted with one or more groups selected from C₁₋₄ alkyl, OR″, CN, OCF₃, F, Cl, Br, I; wherein R″ is H, alkyl, aralkyl, or heteroaralkyl;
R is

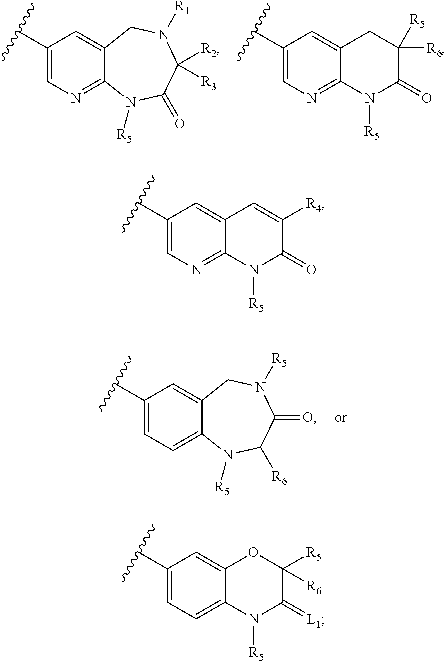

wherein, independently for each occurrence,
R₁ is H, alkyl, or aryl, or R₁ and R₂ taken together form a fused ring;
R₂ is H, alkyl, or aryl, or R₂ and R₁ taken together form a fused ring, or R₂ and R₃ taken together form a spirocyclic ring;
R₃ is H, alkyl, or aryl, or R₃ and R₂ taken together form a spirocyclic ring;
R₄ is H, alkyl, aryl, hydroxy substituted alkyl, or —C(O)ONa;
R₅ is H, alkyl, or aryl;
R₆ is H, alkyl, or aryl; and
L₁ is O or H₂;

In a further embodiment, other compounds useful in the treatment of mastitis (e.g., bovine mastitis) include compounds of formula (XIV) and the attendant definitions, wherein A is selected from the following:

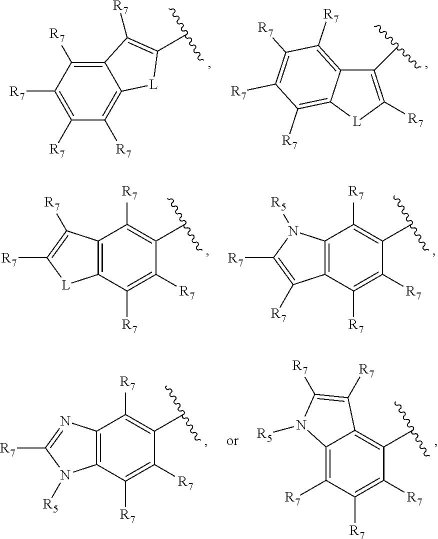

wherein, independently for each occurrence,
R₇ is H, C₁₋₄ alkyl, C₁₋₄ haloalkyl, C₁₋₄ alkenyl, OR″, CN, OCF₃, F, Cl, Br, I; wherein R″ is H, alkyl, aralkyl, or heteroaralkyl;
L is O, S, or NR₅; and
R₅ is as defined previously.

In a further embodiment, other compounds useful in the treatment of bovine mastitis include compounds of formula (XIV) and the attendant definitions, wherein A is selected from the following:

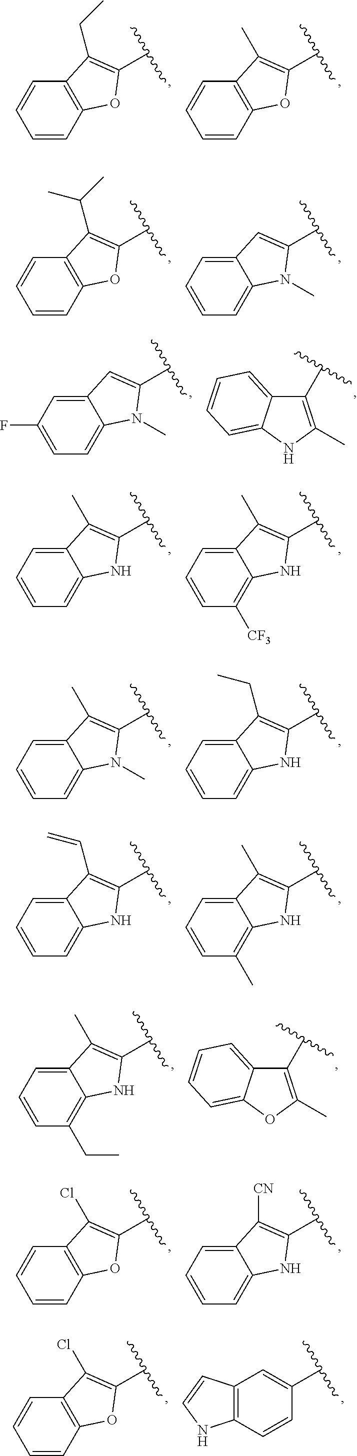

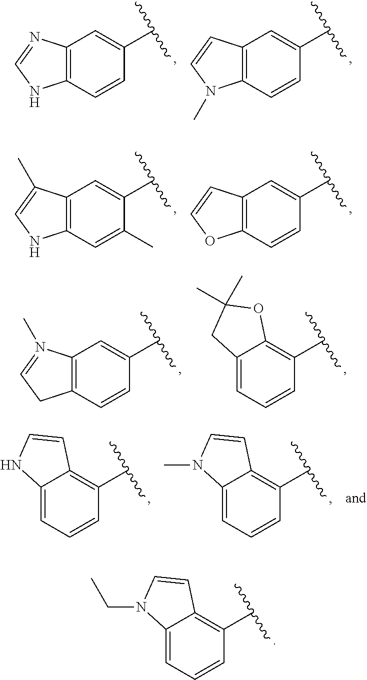

In a further embodiment, other compounds of formula (XIV) useful in the treatment of bovine mastitis are provided, wherein the compound has formula (XIVa):

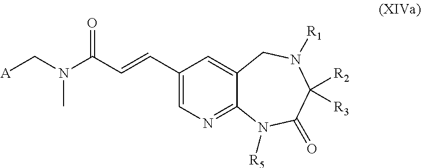

wherein, independently for each occurrence,
R₁, R₂, and R₃ are as previously defined; and

A is selected from the following:

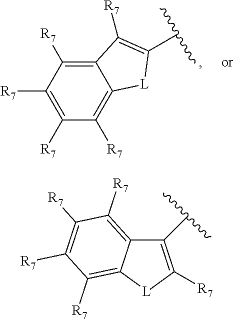

wherein L and R₇ are as previously defined.

In another embodiment, other compounds useful in the treatment of mastitis include compounds of formula (XIVa) and the attendant definitions, wherein R₁ is H.

In another embodiment, other compounds useful in the treatment of mastitis include compounds of formula (XIVa) and the attendant definitions, wherein R₁ is phenyl.

In another embodiment, other compounds useful in the treatment of mastitis include compounds of formula (XIVa) and the attendant definitions, wherein R₂ is methyl and R₃ is methyl.

In another embodiment, other compounds useful in the treatment of mastitis include compounds of formula (XIVa) and the attendant definitions, wherein R₁ and R₂ taken together form a five membered ring.

In another embodiment, other compounds useful in the treatment of mastitis include compounds of formula (XIVa) and the attendant definitions, wherein R₂ and R₃ taken together form a five membered ring.

In another embodiment, other compounds useful in the treatment of mastitis include compounds of formula (XIVa) and the attendant definitions, wherein A is

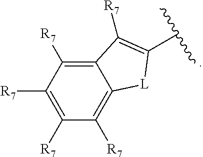

In another embodiment, other compounds useful in the treatment of mastitis include compounds of formula (XIVa) and the attendant definitions, wherein A is

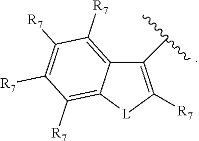

In another embodiment, other compounds useful in the treatment of mastitis include compounds of formula (XIVa) and the attendant definitions, wherein A is

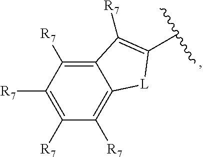

and L is O, and R₇ independently is H, alkyl, or Cl.

In another embodiment, other compounds useful in the treatment of mastitis include compounds of formula (XIVa) and the attendant definitions, wherein A is

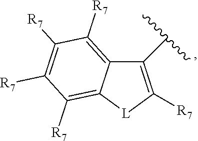

### and L is NH.

In another embodiment, other compounds useful in the treatment of mastitis include compounds of formula (XIVa) and the attendant definitions, wherein A is

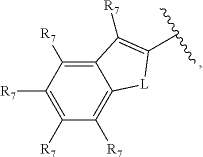

R₁ is phenyl, and R₅ is H.

In a further embodiment, other compounds useful in the treatment of mastitis include compounds of formula (XIV), wherein the compound has formula (XIVb):

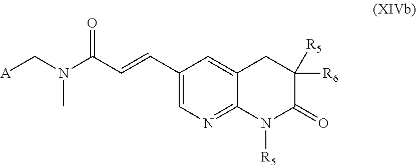

wherein, independently for each occurrence:
A is selected from the following:

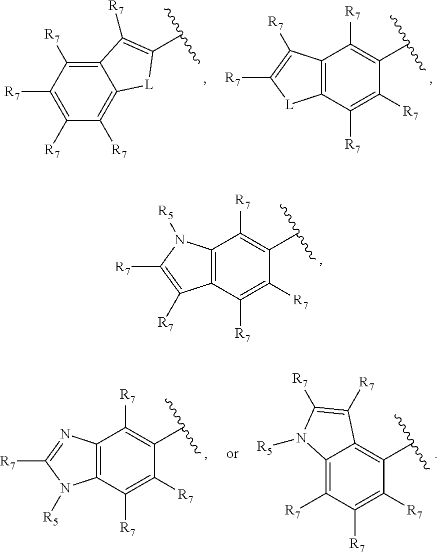

In another embodiment, other compounds useful in the treatment of mastitis include compounds of formula (XIVb) and the attendant definitions, wherein A is

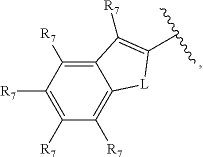

### and L is NH.

In another embodiment, other compounds useful in the treatment of mastitis include compounds of formula (XIVb) and the attendant definitions, wherein A is

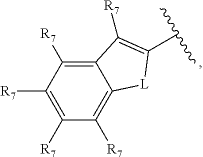

### and L is O.

In another embodiment, other compounds useful in the treatment of mastitis include compounds of formula (XIVb) and the attendant definitions, wherein A is

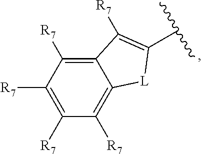

L is NH, and R₇ independently is H, CN, or alkyl.

In another embodiment, other compounds useful in the treatment of mastitis include compounds of formula (XIVb) and the attendant definitions, wherein A is

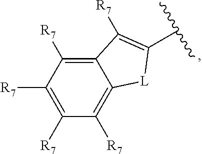

L is O, and R₇ independently is H or Cl.

In another embodiment, other compounds useful in the treatment of mastitis include compounds of formula (XIVb) and the attendant definitions, wherein A is

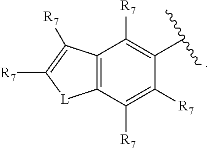

In another embodiment, other compounds useful in the treatment of mastitis include compounds of formula (XIVb) and the attendant definitions, wherein A is

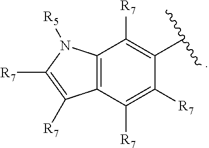

In another embodiment, other compounds useful in the treatment of mastitis include compounds of formula (XIVb) and the attendant definitions, wherein A is

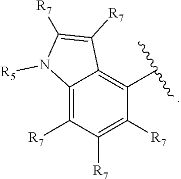

In a further embodiment, other compounds useful in the treatment of mastitis include compounds of formula XIV, wherein the compound has formula (XIVd):

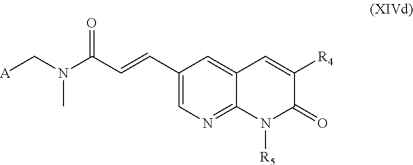

wherein:
A is

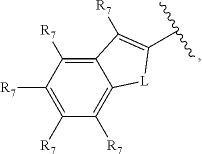

and
L, R₄, R₅, and R₇ are as previously defined.

In another embodiment, other compounds useful in the treatment of mastitis include compounds of formula (XIVd) and the attendant definitions, wherein L is S.

In another embodiment, other compounds useful in the treatment of mastitis include compounds of formula (XIVd) and the attendant definitions, wherein R₇ independently is H or alkyl.

In another embodiment, other compounds useful in the treatment of mastitis include compounds of formula (XIVd) and the attendant definitions, wherein R₄ is H.

In another embodiment, other compounds useful in the treatment of mastitis include compounds of formula (XIVd) and the attendant definitions, wherein R₄ is —C(O)ONa.

A variety of subject compounds and intermediates of them may be made by a person of ordinary skill in the art using conventional reaction techniques. Non-limiting examples of compounds and methods of making them may be found in U.S. patent application Ser. Nos. 08/790,043, 10/009,219, 10/089,019, 09/968,129, 09/968,123, 09/968,236, 09/959,172, 09/979,560, 09/980,369, 10/089,755, 10/089,739, 10/089,740, and PCT Application Nos. PCT/U.S. Ser. No. 03/38706, WO 0027628 and WO 0210332.

Also included are compositions and methods for treating mastitis with pharmaceutically acceptable addition salts and complexes of the disclosed compunds. In cases wherein the inhibitors may have one or more chiral centers, unless specified, this compositions may include each unique racemic compound, as well as each unique nonracemic compound.

In cases in which the inhibitors have unsaturated carbon-carbon double bonds, both the cis (Z) and trans (E) isomers are contemplated. In cases wherein compounds may exist in tautomeric forms, such as keto-enol tautomers, such as

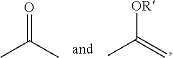

each tautomeric form is contemplated, whether existing in equilibrium or locked in one form by appropriate substitution with R′. The meaning of any substituent at any one occurrence is independent of its meaning, or any other substituent's meaning, at any other occurrence.

Also included are compositions and methods for the treatment of bovine mastitis with prodrugs of the disclosed compounds. Prodrugs are considered to be any covalently bonded carriers which release the active parent drug in vivo.

In some embodiments, the compounds useful for treating bovine mastitis inhibit FabI. Additionally in the treatment of bovine mastitis, the compounds may be useful in combination with known antibiotics.

In some embodiments, contemplated compounds for use in a disclosed methods may have dual FabI/FabK inhibition characteristics and may be useful e.g., as abroad spectrum antibiotics. For example, (E)-N-methyl-N-(1-methyl-1H-indol-3-ylmethyl)-3-(7-oxo-5,6,7,8-tetrahydro-1,8-naphthyridin-3-yl)acrylamide and (E)-N-methyl-N-(2-methyl-1H-indol-3-ylmethyl)-3-(7-oxo-5,6,7,8-tetrahydro-1,8-naphthyridin-3-yl)acrylamide, may have dual FabI/FabK inhibition characteristics.

For example, contemplated methods may include administration of one or more of the following compounds:
(E)-N-methyl-N-((2-methyl-1H-indol-3-yl)methyl)-3-(7-oxo-5,6,7,8-tetrahydro-1,8-naphthyridin-3-yl)acrylamide;
(E)-N-methyl-N-((3-methylbenzofuran-2-yl)methyl)-3-(2,3,4,5-tetrahydro-1H-pyrido[2,3-e][1,4]diazepin-7-yl)acrylamide;
(E)-N-methyl-N-(3-methylbenzofuran-2-ylmethyl)-3-(7-oxo-5,6,7,8-tetrahydro-1,8-naphthyridin-3-yl)acrylamide;
(E)-3-(6-aminopyridin-3-yl)-N-(4,6-dichloro-1-methyl-1H-indol-2-ylmethyl)-N-methylacrylamide;
(E)-3-(2-aminopyrimidin-5-yl)-N-(2-methyl-1H-indol-3-ylmethyl)-N-methylacrylamide;
(E)-3-(6-aminopyridin-3-yl)-N-(1-ethyl-1H-indol-3-ylmethyl)-N-methylacrylamide;
(E)-3-(6-aminopyridin-3-yl)-N-(1-isopropyl-1H-indol-3-ylmethyl)-N-methylacrylamide;
(E)-N-methyl-N-(1-methyl-1H-indol-3-ylmethyl)-3-[6-(pyridin-2-ylamino)pyridin-3-yl]acrylamide;
(E)-3-(6-aminopyridin-3-yl)-N-(1,4-dimethyl-1H-indol-3-ylmethyl)-N-methylacrylamide;
(E)-3-(6-aminopyridin-3-yl)-N-(3,3 -dimethyl-3H-indene-1-ylmethyl)-N-methylacrylamide;
(E)-3-(2-aminopyrimidin-5-yl)-N-methyl-N-(1-methyl-1H-pyrrolo[2,3-b]pyridin-3-ylmethyl)acrylamide;
(E)-N-methyl-N-(1-methyl-1H-pyrrolo[2,3-b]pyridin-3-ylmethyl)-3-(7-oxo-5,6,7,8-tetrahydro-1,8-naphthyridin-3-yl)acrylamide;
(E)-N-methyl-N-(2-methylbenzo[b]thiophen-3-ylmethyl)-3-(7-oxo-5,6,7,8-tetrahydro-1,8-naphthyridin-3-yl)acrylamide;
(E)-N-methyl-N-(2-methyl-1H-indol-3-ylmethyl)-3-(3-methyl-2-oxo-1,2,3,4-tetrahydropyrido[2,3-d]pyrimidin-6-yl)acrylamide;
(E)-3-(3H-imidazo[4,5-b]pyridin-6-yl)-N-methyl-N-(1-methyl-1H-indol-3-ylmethyl)acrylamide;
(E)-3-(3,4-dihydro-2H-pyrido[3,2-b]-1,4-oxazin-7-yl)-N-methyl-N-(1-methyl-1H-indol-3-ylmethyl)acrylamide;
(E)-N-(1H-indol-3-ylmethyl)-N-methyl-3-(7-oxo-5,6,7,8-tetrahydro-1,8-naphthyridin-3-yl)acrylamide;
(E)-3-(6-aminopyridin-3-yl)-N-(5-methoxy-1-methyl-1H-indol-3-ylmethyl)-N-methylacrylamide;
(E)-3-(6-aminopyridin-3-yl)-N-(4-methoxy-1-methyl-1H-indol-3-ylmethyl)-N-methylacrylamide;
(E)-3-(6-aminopyridin-3-yl)-N-(1H-indol-3-ylmethyl)-N-methylacrylamide
(E)-3-(6-aminopyridin-3-yl)-N-(7 -chloro-1-methyl-1H-indol-3-ylmethyl)-N-methylacrylamide;
(E)-N-methyl-N-(1-methyl-1H-indol-2-ylmethyl)-3-[6-[N-(methylaminocarbonylmethyl)amino]pyridin-3-yl]acrylamide;
(E)-3-(6-amino-5-(methoxycarbonyl)pyridin-3-yl)-N-(1-methyl-1H-indol-3-ylmethyl)-N-methylacrylamide;
(E)-N-(1-benzyl-1H-indol-3-ylmethyl)-N-methyl-3-(7-oxo-5,6,7,8-tetrahydro-1,8-naphthyridin-3-yl)acrylamide;
(E)-3-(6-aminopyridin-3-yl)-N-(7-methoxy-1-methyl-1H-indol-3-ylmethyl)-N-methylacrylamide;
(E)-N-methyl-N-(1-methyl-1H-indol-3-ylmethyl)-3-(3-methyl-2-oxo-1,2,3,4-tetrahydropyrido[2,3-d]pyrimidin-6-yl)acrylamide;
(E)-N-methyl-3-(7 -oxo-5,6,7,8-tetrahydro-1,8-naphthyridin-3-yl)-N-(1,2,7-trimethyl-1H-indol-3-ylmethyl)acrylamide;
(E)-N-[1-(2-dimethylaminoethyl)-1H-indol-3-ylmethyl]-N-methyl-3-(7-oxo-5,6,7, 8-tetrahydro-1,8-naphthyridin-3-yl)acrylamide;
(E)-N-(7-chloro-1-methyl-1H-indol-3-ylmethyl)-N-methyl-3-(7-oxo-5,6,7,8-tetrahydro-1,8-naphthyridin-3-yl)acrylamide;
(E)-3-[6-amino-5-[[N-methyl-N-(2-methyl-1H-indol-3-ylmethyl)amino]carbonylethyl]pyridin-3-yl]-N-methyl-N-(2-methyl-1H-indol-3-ylmethyl)acrylamide;
(E)-N-(2,3-dihydro-1H-3 a-azacyclopenta[a]indene-8-ylmethyl)-N-methyl-3-(7-oxo-5,6,7,8-tetrahydro-1,8-naphthyridin-3-yl)acrylamide;
(E)-N-methyl-N-(2-methyl-1H-indol-3-ylmethyl)-3-(2-oxo-2,3-dihydro-1H-pyrrolo[2,3-b]pyridin-5-yl)acrylamide;
(E)-N-(1-ethyl-5-fluoro-1H-indol-3-ylmethyl)-N-methyl-3-(7-oxo-5,6,7,8-tetrahydro-1,8-naphthyridin-3-yl)acrylamide;
(E)-N-(7-chloro-1H-indol-3-ylmethyl)-N-methyl-3-(7-oxo-5,6,7,8-tetrahydro-1,8-naphthyridin-3-yl)acrylamide;
(E)-3-(6-aminopyridin-3-yl)-N-(6-chloro-1-methyl-1H-indol-3-ylmethyl)-N-methylacrylamide;
(E)-N-(5-fluoro-1-methyl-1H-indol-3-ylmethyl)-N-methyl-3-(7-oxo-5,6,7,8-tetrahydro-1,8-naphthyridin-3-yl)acrylamide;
(E)-N-(6-fluoro-1-methyl-1H-indol-3-ylmethyl)-N-methyl-3-(7-oxo-5,6,7,8-tetrahydro-1,8-naphthyridin-3-yl)acrylamide;
(E)-N-(7-fluoro-1-methyl-1H-indol-3-ylmethyl)-N-methyl-3-(7-oxo-5,6,7,8-tetrahydro-1,8-naphthyridin-3-yl)acrylamide;
(E)-3-(6-aminopyridin-3-yl)-N-(7-hydroxy-1-methyl-1H-indol-3-ylmethyl)-N-methylacrylamide;
(E)-3-(6-aminopyridin-3-yl)-N-(6-fluoro-1-methyl-1H-indol-3-ylmethyl)-N-methylacrylamide;
(E)-3-(6-aminopyridin-3-yl)-N-(5-chloro-1-methyl-1H-indol-3-ylmethyl)-N-methylacrylamide;
(E)-3-(6-aminopyridin-3-yl)-N-(4-chloro-1-methyl-1H-indol-3-ylmethyl)-N-methylacrylamide;
(E)-N-methyl-3-(8-oxo-6,7,8,9-tetrahydro-5H-pyrido[2,3-b]azepin-3-yl)acrylamide;
(E)-3-[6-[N-(methoxycarbonylmethyl)amino]pyridin-3-yl]-N-methyl-N-(2-methyl-1H-indol-3-ylmethyl)acrylamide;
(E)-3-[6-[N-(carboxymethyl)amino]pyridin-3-yl]-N-methyl-N-(2-methyl-1H-indol-3-ylmethyl)acrylamide;
(E)-N-methyl-N-(2-methyl-1H-indol-3-ylmethyl)-3-[6-[N-(methylaminocarbonylmethyl)amino]pyridin-3-yl]acrylamide;
(E)-N-(7-chloro-1-methyl-1H-indol-3-ylmethyl)-3 -[6-[N-(methoxycarbonylmethyl)amino]pyridin-3-yl]-N-methylacrylamide;
(E)-2,N-dimethyl-N-(2-methyl-1H-indol-3-ylmethyl)-3 -(7-oxo-5,6,7 ,8-tetrahydro-1,8-naphthyridin-3 -yl)acrylamide;
(E)-3-[6-[N-(carboxymethyl)amino]pyridin-3-yl]-N-(7-chloro-1-methyl-1H-indol-3-ylmethyl)-N-methylacrylamide;
(E)-N-(7-chloro-1-methyl-1H-indol-3-ylmethyl)-N-methyl-3-[6-[N-(methylaminocarbonylmethyl)amino]pyridin-3-yl]acrylamide;
(E)-3,N-dimethyl-N-(2-methyl-1H-indol-3-ylmethyl)-3-(7-oxo-5,6,7,8-tetrahydro-1,8-naphthyridin-3-yl)acrylamide;
(E)-3-(6-aminopyridin-3-yl)-N-(4-fluoro-1-methyl-1H-indol-3-ylmethyl)-N-methylacrylamide;
(E)-3-(6-aminopyridin-3-yl)-N-(5-fluoro-1-methyl-1H-indol-3-ylmethyl)-N-methylacrylamide;
(E)-3-(6-aminopyridin-3-yl)-N-(7-fluoro-1-methyl-1H-indol-3-ylmethyl)-N-methylacrylamide;
(E)-3-(6-aminopyridin-3-yl)-N-(7-methoxycarbonyl-1-methyl-1H-indol-3-ylmethyl)-N-methylacrylamide;
(E)-3-(6-aminopyridin-3-yl)-N-(7-fluoro-1H-indol-3-ylmethyl)-N-methylacrylamide;
(E)-3-(6-aminopyridin-3-yl)-N-methyl-N-(1,2,7-trimethyl-1H-indol-3-ylmethyl)acrylamide;
(E)-N-methyl-N-(2-methyl-1H-indol-3-ylmethyl)-3-(8-oxo-6,7 ,8,9-tetrahydro-5H-pyrido[2,3-b]azepin-3-yl)acrylamide;
(E)-3-(6-aminopyridin-3-yl)-N-(7-chloro-1H-indol-3-ylmethyl)-N-methylacrylamide;
(E)-3-(6-aminopyridin-3-yl)-N-(2-chloro-1-methyl-1H-indol-3-ylmethyl)-N-methylacrylamide;
(E)-N-(2-chloro-1-methyl-1H-indol-3-ylmethyl)-N-methyl-3-(7-oxo-5,6,7 ,8-tetrahydro-1,8-naphthyridin-3-yl)acrylamide;
(E)-N-(5-chloro-1-methyl-1H-indol-3-ylmethyl)-N-methyl-3-(7-oxo-5,6,7,8-tetrahydro-1,8-naphthyridin-3-yl)acrylamide;
(E)-N-(4-fluoro-1-methyl-1H-indol-3-ylmethyl)-N-methyl-3-(7-oxo-5,6,7,8-tetrahydro-1,8-naphthyridin-3-yl)acrylamide;
(E)-N-(6-chloro-1-methyl-1H-indol-3-ylmethyl)-N-methyl-3-(7-oxo-5,6,7,8-tetrahydro-1,8-naphthyridin-3-yl)acrylamide;
(E)-3-(6-aminopyridin-3-yl)-N-(4-fluoro-1H-indol-3-ylmethyl)-N-methylacrylamide;
(E)-3-(6-aminopyridin-3-yl)-N-(7-carboxy-1-methyl-1H-indol-3-ylmethyl)-N-methylacrylamide;
(E)-N-(1,7-dimethyl-1H-indol-3-ylmethyl)-N-methyl-3-(7-oxo-5,6,7 ,8-tetrahydro-1,8-naphthyridin-3 -yl)acrylamide;
(E)-N-(1,6-dimethyl-1H-indol-3-ylmethyl)-N-methyl-3-(7-oxo-5,6,7,8-tetrahydro-1,8-naphthyridin-3-yl)acrylamide;
(E)-N-(1,4-dimethyl-1H-indol-3-ylmethyl)-N-methyl-3-(7-oxo-5,6,7,8-tetrahydro-1,8-naphthyridin-3 -yl)acrylamide;
(E)-N-(3,3-dimethyl-3H-indene-1-ylmethyl)-N-methyl-3-(7-oxo-5,6,7,8-tetrahydro-1,8-naphthyridin-3-yl)acrylamide;
(E)-N-(1,5-dimethyl-1H-indol-3-ylmethyl)-N-methyl-3-(7-oxo-5,6,7,8-tetrahydro-1,8-naphthyridin-3 -yl)acrylamide;
(E)-N-(7-methoxy-1-methyl-1H-indol-3-ylmethyl)-N-methyl-3-(7-oxo-5,6,7,8-tetrahydro-1,8-naphthyridin-3-yl)acrylamide;
(E)-N-(7-hydroxy-1-methyl-1H-indol-3-ylmethyl)-N-methyl-3-(7-oxo-5,6,7,8-tetrahydro-1,8-naphthyridin-3-yl)acrylamide;
N-Methyl-N-(2-methyl-1H-indol-3-ylmethyl)-3-(4-methyl-3-oxo-2,3,4,5-tetrahydro-1H-pyrido[2,3-e]-1,4-diazepin-7-yl)acrylamide;
(E)-N-[1-(2-hydroxyethyl)-1H-indol-3-ylmethyl]-N-methyl-3-(7-oxo-5,6,7,8-tetrahydro-1,8-naphthyridin-3-yl)acrylamide;
(E)-N-(4-chloro-1-methyl-1H-indol-3-ylmethyl)-N-methyl-3-(7-oxo-5,6,7,8-tetrahydro-1,8-naphthyridin-3-yl)acrylamide;
(E)-N-methyl-N-(1-methyl-1H-indol-3-ylmethyl)-3-(8-oxo-6,7,8,9-tetrahydro-5H-pyrido[2,3-h]azepin-3-yl)acrylamide;
(E)-N-(4-methoxy-1-methyl-1H-indol-3-ylmethyl)-N-methyl-3-(7-oxo-5,6,7,8-tetrahydro-1,8-naphthyridin-3-yl)acrylamide;
(E)-N-(5-methoxy-1-methyl-1H-indol-3-ylmethyl)-N-methyl-3-(7-oxo-5,6,7,8-tetrahydro-1,8-naphthyridin-3-yl)acrylamide;
(E)-N-(6-methoxy-1-methyl-1H-indol-3-ylmethyl)-N-methyl-3-(7-oxo-5,6,7,8-tetrahydro-1,8-naphthyridin-3-yl)acrylamide;
(E)-N-(naphthalen-2-ylmethyl)-N-methyl-3-(7-oxo-5,6,7,8-tetrahydro-1,8-naphthyridin-3-yl)acrylamide;
(E)-N-(quinolin-3-ylmethyl)-N-methyl-3-(7-oxo-5,6,7,8-tetrahydro-1,8-naphthyridin-3-yl)acrylamide;
(E)-N-(1-methyl-1H-indol-3-ylmethyl)-N-methyl-3-(6-amino-7-oxo-5,6,7,8-tetrahydro-1,8-naphthyridin-3-yl)acrylamide;
(E)-N-(1-ethyl-1H-indol-3-ylmethyl)-N-methyl-3-(7-oxo-5,6,7,8-tetrahydro-1,8-naphthyridin-3-yl)acrylamide;
(E)-N-(naphthalen-1-ylmethyl)-N-methyl-3-(7-oxo-5,6,7,8-tetrahydro-1,8-naphthyridin-3-yl)acrylamide;
(E)-N-(benzofuran-2-ylmethyl)-N-methyl-3-(7-oxo-5,6,7,8-tetrahydro-1,8-naphthyridin-3-yl)acrylamide;
(E)-3-(6-aminopyridin-3-yl)-N-(6-methoxycarbonyl-1-methyl-1H-indol-3-ylmethyl)-N-methylacrylamide;
(E)-N-methyl-N-(2-methyl-1H-indol-3-ylmethyl)-3-[3-(2-methoxyethyl)-2-oxo-1,2,3,4-tetrahydropyrido[2,3-d]pyrimidin-6-yl]acrylamide;
(E)-N-(1-methyl-1H-indol-3-ylmethyl)-N-methyl-3-[6-(methoxycarbonyl)-7-oxo-5,6,7,8-tetrahydro-1,8-naphthyridin-3-yl]acrylamide;
(E)-3-(6-aminopyridin-3-yl)-N-(1,3-dimethyl-1H-pyrrolo[2,3-b]pyridin-3-ylmethyl)-N-methylacrylamide;
(E)-N-(1,3-dimethyl-1H-pyrrolo[2,3-b]pyridin-3-ylmethyl)-N-methyl-3-(7-oxo-5,6,7,8-tetrahydro-1,8-naphthyridin-3-yl)acrylamide;
(E)-3-(6-aminopyridin-3-yl)-N-methyl-N-(1-methyl-1H-pyrrolo[2,3-c]pyridin-3-ylmethyl)acrylamide;
(E)-3-(6-aminopyridin-3-yl)-N-methyl-N-(1-methyl-1H-pyrrolo[3,2-c]pyridin-3-ylmethyl)acrylamide;
(E)-3-(6-aminopyridin-3-yl)-N-methyl-N-(1-methyl-1H-pyrrolo[3,2-b]pyridin-3-ylmethyl)acrylamide;
(E)-N-methyl-N-(1-methyl-1H-pyrrolo[2,3-c]pyridin-3-ylmethyl)-3-(7-oxo-5,6,7,8-tetrahydro-1,8-naphthyridin-3-yl)acrylamide;
(E)-N-methyl-N-(1-methyl-1H-pyrrolo[3,2-c]pyridin-3-ylmethyl)-3-(7-oxo-5,6,7,8-tetrahydro-1,8-naphthyridin-3-yl)acrylamide;
(E)-N-methyl-N-(1-methyl-1H-pyrrolo[3,2-b]pyridin-3-ylmethyl)-3-(7-oxo-5,6,7,8-tetrahydro-1,8-naphthyridin-3-yl)acrylamide;
(E)-3-(6-aminopyridin-3-yl)-N-(benzofuran-3 -ylmethyl)-N-methylacrylamide;
(E)-3-(6-aminopyridin-3-yl)-N-methyl-N-(3-methylbenzofuran-2-ylmethyl)acrylamide;
(E)-3-(6-aminopyridin-3-yl)-N-methyl-N-(2-methylbenzofuran-3-ylmethyl)acrylamide;
(E)-N-(benzofuran-3-ylmethyl)-N-methyl-3-(7-oxo-5,6,7,8-tetrahydro-1,8-naphthyridin-3-yl)acrylamide;
(E)-N-methyl-N-(3-methylbenzofuran-2-ylmethyl)-3-(7-oxo-5,6,7,8-tetrahydro-1,8-naphthyridin-3-yl)acrylamide;
(E)-N-methyl-N-(2-methylbenzofuran-3-ylmethyl)-3-(7-oxo-5,6,7,8-tetrahydro-1,8-naphthyridin-3-yl)acrylamide;
(E)-(6-aminopyridin-3-yl)-N-methyl-N-[1-(1-methyl-1H-indol-2-yl)ethyl]acrylamide;
(E)-(6-aminopyridin-3-yl)-N-methyl-N-[1-(1-methyl-1H-indol-3-yl)ethyl]acrylamide;
(E)-N-methyl-N-[1-(1-methyl-1H-indol-2-yl)ethyl]-3-(7-oxo-5,6,7,8-tetrahydro-1,8-naphthyridin-3-yl)acrylamide; and
(E)-N-methyl-N-[1-(1-methyl-1H-indol-3-yl)ethyl]-3-(7-oxo-5,6,7,8-tetrahydro-1,8-naphthyridin-3-yl)acrylamide;
or a pharmaceutically acceptable salt thereof.

Representative of the novel compounds contemplated herein are the following:
(2S)-2-[(carbomethoxy)methyl]-N,4-dimethyl-N-[(1-methyl-1H-indol-2-yl)methyl]-3-oxo-2,3,4,5-tetrahydro-1H-1,4-benzodiazepine-7-carboxamide;
(2R)-2-[(carbomethoxy)methyl]-N,4-dimethyl-N-[(1-methyl-1H-indol-2-yl)methyl]-3-oxo-2,3,4,5-tetrahydro-1H-1,4-benzodiazepine-7-carboxamide;
(2R)-N-[(1-methyl-1H-indol-2-yl)methyl]-3-oxo-N,2,4-trimethyl-2,3,4,5-tetrahydro-1H-1,4-benzodiazepine-7-carboxamide;
(2R)-2-benzyl-N,4-dimethyl-N-[(1-methyl-1H-indol-2-yl)methyl]-3-oxo-2,3,4,5-tetrahydro-1H-1,4-benzodiazepine-7-carboxamide;
(2R)-2-[(carbomethoxy)methyl]-N,4-dimethyl-N-[[1-(4-hydroxybenzyl)-1H-indol-2-yl]methyl]-3-oxo-2,3,4,5-tetrahydro-1H-1,4-benzodiazepine-7-carboxamide;
(2R)-N-[[1-(4-hydroxybenzyl)-1H-indol-2-yl]methyl]-3-oxo-N,2,4-trimethyl-2,3,4,5-tetrahydro-1H-1,4-benzodiazepine-7-carboxamide;
(2R)-N,4-dimethyl-2-(hydroxymethyl)-N-R1-methyl-1H-indol-2-yl)methyl]-3-oxo-2,3,4,5-tetrahydro-1H-1,4-benzodiazepine-7-carboxamide;
N,4-dimethyl-N-[(1-methyl-1H-indol-2-yl)methyl]-3-oxo-2,3,4,5-tetrahydro-1H-1,4-benzodiazepine-7-carboxamide;
(2R)-N,4-dimethyl-N-[[1-(4-hydroxybenzyl)-1H-indol-2-yl]methyl]-2-(hydroxymethyl)-3-oxo-2,3,4,5-tetrahydro-1H-1,4-benzodiazepine-7-carboxamide;
(2R)-N,4-dimethyl-N-[(5-benzyloxy-1-methyl-1H-indol-2-yl)methyl]-2-(hydroxymethyl)-3-oxo-2,3,4,5-tetrahydro-1H-1,4-benzodiazepine-7-carboxamide;
(2R)-N,4-dimethyl-N-2-(hydroxymethyl)-[(5-hydroxy-1-methyl-1H-indol-2-yl)methyl]-3-oxo-2,3,4,5-tetrahydro-1H-1,4-benzodiazepine-7-carboxamide;
(2R)-N,4-dimethyl-N-[(1-methyl-1H-indol-2-yl)methyl]-3-oxo-2-propyl-2,3,4,5-tetrahydro-1H-1,4-benzodiazepine-7-carboxamide;
(2R)-4-benzyl-2-(hydroxymethyl)-N-methyl-N-R1-methyl-1H-indol-2-yl)methyl]-3-oxo-2,3,4,5-tetrahydro-1H-1,4-benzodiazepine-7-carboxamide; and
(2R)-2-(hydroxymethyl)-N-methyl-N-[(1-methyl-1H-indol-2-yl)methyl]-3-oxo-4-phenethyl-2,3,4,5-tetrahydro-1H-1,4-benzodiazepine-7-carboxamide;
or pharmaceutically acceptable salts thereof.

Representative compounds useful for treatment of bovine mastitis are the following compounds:
(E)-3-(6-Aminopyridin-3-yl)-N-methyl-N-(1-methyl-1H-indol-2-ylmethyl)acrylamide;
(E)-3-(4-Aminophenyl)-N-methyl-N-(1-methyl-1H-indol-2-ylmethyl)acrylamide;
(E)-N-Methyl-N-(1-methyl-1H-indol-2-ylmethyl)-3-(pyridin-3-yl)acrylamide;
(E)-3-(2-Aminopyrimidin-5-yl)-N-methyl-N-(1-methyl-1H-indol-2-ylmethyl)acrylamide;
(E)-3-(6-Aminopyridin-3-yl)-N-(benzo[b]thiophen-2-ylmethyl)-N-methylacrylamide;
(E)-3-(6-Aminopyridin-3-yl)-N-methyl-N-(1-methyl-1H-indol-2-ylmethyl)-2-butenamide;
(E)-3-(6-Aminopyridin-3-yl)-N-methyl-N-(1-methyl-1H-indazol-3-ylmethyl)acrylamide;
(E)-3-(6-Amino-2-methylpyridin-3-yl)-N-methyl-N-(1-methyl-1H-indol-2-ylmethyl)acrylamide;
(E)-3-(6-Amino-5-methylpyridin-3-yl)-N-methyl-N-(1-methyl-1H-indol-2-ylmethyl)acrylamide;
(E)-3-(6-Aminopyridin-3-yl)-N-(1-methyl-1H-indol-2-ylmethyl)-N-propylacrylamide;
(E)-3-(6-Aminopyridin-3-yl)-N-(5-fluoro-1-methyl-1H-indol-2-ylmethyl)-N-methylacrylamide;
(E)-3-(6-Aminopyridin-3-yl)-N-methyl-N-(naphthalen-1-ylmethyl)acrylamide;
(E)-3-(6-Aminopyridin-3-yl)-2,N-dimethyl-N-(1-methyl-1H-indol-2-ylmethyl)acrylamide;
(E)-3-(6-Aminopyridin-3-yl)-N-methyl-N-(naphthalen-2-ylmethyl)acrylamide; 2-(6-Aminopyridin-3-ylmethyl)-N-methyl-N-(1-methyl-1H-indol-2-ylmethyl)acrylamide;
(E)-3-(6-Aminopyridin-3-yl)-N-(benzofuran-2-ylmethyl)-N-methylacrylamide;
(E)-3-(3,4-Dihydro-2H-pyrido[3,2-b]-1,4-oxazin-7-yl)-N-methyl-(1-methyl-1H-indol-2-ylmethyl)acrylamide;
(E)-N-Methyl-3-[6-(methylamino)pyridin-3-yl]-N-(1-methyl-1H-indol-2-ylmethyl)acrylamide;
(E)-3-[6-(Dimethylamino)pyridin-3-yl]-N-methyl-N-(1-methyl-1H-indol-2-ylmethyl)acrylamide;
(E)-N-Methyl-N-[(1-methyl-1H-indol-2-yl)methyl]-3-(5,6,7,8-tetrahydro-1,8-naphthyridin-3-yl)acrylamide;
(E)-3-[6-(Acetylamino)pyridin-3-yl]-N-methyl-N-(1-methyl-1H-indol-2-ylmethyl)acrylamide;
(E)-3-(6-Amino-5-methylpyridin-3-yl)-N-(benzo[b]thiophen-2-ylmethyl)-N-methylacrylamide;
(E)-3-(6-Amino-5-methylpyridin-3-yl)-N-methyl-N-(naphthalen-2-ylmethyl)acrylamide;
(E)-3-(6-Amino-4-methylpyridin-3-yl)-N-methyl-N-(1-methyl-1H-indol-2-ylmethyl)acrylamide;
(E)-3-(6-Aminopyridin-3-yl)-N-cyclopropyl-N-(1-methyl-1H-indol-2-ylmethyl)acrylamide;
(E)-3-(6-Aminopyridin-3-yl)-N-methyl-N-(1-methyl-1H-indol-3-ylmethyl)acrylamide;
(E)-3-(6-Aminopyridin-3-yl)-N-methyl-N-(quinolin-3-ylmethyl)acrylamide;
(E)-3-(6-Aminopyridin-3-yl)-N-methyl-N-(thieno[2,3-b]thiophen-2-ylmethyl)acrylamide;
(E)-N-Methyl-N-(1-methyl-1H-indol-2-ylmethyl)-3-(6-methylpyridine-3-yl)acrylamide;
(E)-3-[6-(Acetylamino)-5-methylpyridin-3-yl]-N-methyl-N-(1-methyl-1H-indol-2-ylmethyl)acrylamide;
(E)-3-(6-Aminopyridin-3-yl)-N-(1H-indol-2-ylmethyl)-N-methylacrylamide;
(E)-N-Methyl-N-(1-methyl-1H-indol-2-ylmethyl)-3-[6-(2-oxopropylamino)pyridin-3-yl]acrylamide;
(E)-3-(6-Aminopyridin-3-yl)-N-methyl-N-(thieno[3,2-b]thiophen-2-ylmethyl)acrylamide;
(E)-3-[6-Amino-5-(hydroxymethyl)pyridin-3-yl]-N-methyl-N-(1-methyl-1H-indol-2-ylmethyl)acrylamide;
(E)-3-(3H-Imidazo[4,5-b]pyridin-6-yl)-N-methyl-N-(1-methyl-1H-indol-2-ylmethyl)acrylamide;
(E)-3-[6-Aminopyridin-3-yl]-N-(1-ethyl-1H-indol-2-ylmethyl)-N-methylacrylamide;
(E)-3-[6-Aminopyridin-3-yl]-N-(1,3-dimethyl-1H-indol-2-ylmethyl)-N-methylacrylamide;
(E)-3-[6-((E)-But-2-enoylamino)pyridin-3-yl]-N-methyl-N-(1-methyl-1H-indol-2-ylmethyl)acrylamide;
(E)-N-Methyl-N-(1-methyl-1H-indol-2-ylmethyl)-3-(7-oxo-5,6,7,8-tetrahydro-1,8-naphthyridin-3-yl)acrylamide;
(E)-3-[6-Amino-5-[(2-hydroxyethylamino)carbonyl]pyridin-3-yl]-N-(1-methyl-1H-indol-2-ylmethyl)-N-methylacrylamide;
(E)-3-[6-Aminopyridin-3-yl]-N-methyl-N-(3-methyl-1H-inden-2-ylmethyl)acrylamide;
(E)-3-[6-Aminopyridin-3-yl]-N-(1H-inden-2-ylmethyl)-N-methylacrylamide;
(E)-3-(6-Aminopyridin-3-yl)-N-methyl-N-(6-methyl-6H-thieno[2,3-b]pyrrol-5-ylmethyl)acrylamide;
(E)-N-Methyl-N-(1-methyl-1H-indol-2-ylmethyl)-3-(2-oxo-1,4-dihydro-2H-pyrido[2,3-d]-1,3-oxazin-6-yl)acrylamide;
(E)-3-[6-(1,3-dioxo-1,3-dihydroisoindol-2-yl)pyridin-3-yl]-N-methyl-N-(1-methyl-1H-indol-2-ylmethyl)acrylamide;
(E)-3-[6-[(2-Carboxybenzoyl)amino]pyridin-3-yl]-N-methyl-N-(1-methyl-1H-indol-2-ylmethyl)acrylamide;
(E)-3-[6-(3-Ethylureido)pyridin-3-yl]-N-methyl-N-(1-methyl-1H-indol-2-ylmethyl)acrylamide;
(E)-N-(1,3-Dimethyl-1H-indol-2-ylmethyl)-N-methyl-3-(7-oxo-5,6,7,8-tetrahydro-1,8-naphthyridin-3-yl)acrylamide;
(E)-3-(6-Aminopyridin-3 -yl)-N-methyl-N-(3-methylbenzo[b]thiophen-2-ylmethyl)acrylamide;
(E)-3-(6-Aminopyridin-3-yl)-N-(4-methoxy-1-methyl-1H-indol-2-ylmethyl)-N-methylacrylamide;
(E)-3-[6-(Acetylamino)pyridin-3-yl]-N-methyl-N-(3-methyl-1H-inden-2-ylmethyl)acrylamide;
(E)-3-[6-(Acetylamino)pyridin-3-yl]-N-methyl-N-(1-methyl-1H-indol-3-ylmethyl)acrylamide;
(E)-N-Methyl-N-(1-methyl-1H-indol-2-ylmethyl)-3-(3-methyl-2-oxo-1,2,3,4-tetrahydropyrido[2,3-d]pyrimidin-6-yl)acrylamide;
(E)-N-Methyl-N-(1-methyl-1H-indol-2-ylmethyl)-3-[6-(propionylamino)pyridin-3-yl]acrylamide;
(E)-3-(6-Aminopyridin-3-yl)-N-(1,4-dimethyl-1H-indol-2-ylmethyl)-N-methylacrylamide;
(E)-N-methyl-N-(1-methyl-1H-indol-2-ylmethyl)-3-[6-(3-methylureido)pyridin-3-yl]acrylamide;
(E)-N-Methyl-N-(3-methyl-1H-inden-2-ylmethyl)-3-(7-oxo-5,6,7,8-tetrahydro-1,8-naphthyridin-3-yl)acrylamide;
(E)-3-(6-Aminopyridin-3-yl)-N-methyl-N-(4-methyl-4H-theino[3,2-b]pyrrol-5-ylmethyl)acrylamide;
(E)-3-(6-Aminopyridin-3-yl)-N-(3,4-dimethylthieno[2,3-b]thiophen-2-ylmethyl)-N-methylacrylamide;
(E)-N-Methyl-N-(1-methyl-1H-indol-2-ylmethyl)-3-[6-(phenylamino)pyridin-3-yl]acrylamide;
(E)-3-(6-Aminopyridin-3-yl)-N-(6-methoxy-1-methyl-1H-indol-2-ylmethyl)-N-methylacrylamide;
(E)-3-(2-Aminopyrimidin-5-yl)-N-(benzo[b]thiophen-2-ylmethyl)-N-methylacrylamide;
(E)-3-(2-Aminopyrimidin-5-yl)-N-methyl-N-(1-methyl-1H-indol-3-ylmethyl)acrylamide;
(E)-3-(6-Aminopyridin-3-yl)-N-methyl-N-(1-methylnaphthalen-2-ylmethyl)acrylamide;
(E)-3-(6-Aminopyridin-3-yl)-N-(1,2-dimethyl-1H-indol-3-ylmethyl)-N-methylacrylamide;
(E)-3-(6-Aminopyridin-3 -yl)-N-(benzo[b]thiophen-3-ylmethyl)-N-methylacrylamide;
(E)-N-Methyl-N-(1-methyl-1H-indol-3-ylmethyl)-3-(7-oxo-5,6,7,8-tetrahydro-1,8-naphthyridin-3-yl)acrylamide;
(E)-3-[2-Aminopyrimidin-5-yl]-N-methyl-N-(3-methyl-1H-inden-2-ylmethyl)acrylamide;
(E)-N-Methyl-N-(1-methyl-1H-indol-2-ylmethyl)-3-[6-(pyridin-2-ylamino)pyridin-3-yl]acrylamide;
(E)-3-[2-(Acetylamino)pyrimidin-5-yl]-N-methyl-N-(1-methyl-1H-indol-2-ylmethyl)acrylamide;
(E)-3-(6-Aminopyridin-3-yl)-N-methyl-N-(2-methyl-1H-indol-3-ylmethyl)acrylamide;
(E)-3-(2-Aminopyrimidin-5-yl)-N-(1,2-dimethyl-1H-indol-3-ylmethyl)-N-methylacrylamide;
(E)-N-(1,2-Dimethyl-1H-indol-3-ylmethyl)-N-methyl-3-(7-oxo-5,6,7,8-tetrahydro-1,8-naphthyridin-3-yl)acrylamide;
(E)-N-Methyl-N-(1-methyl-1H-indol-2-ylmethyl)-3-(3-oxo-3,4-dihydro-2H-pyrido[3,2-b]-1,4-oxazin-7-yl)acrylamide;
(E)-N-Methyl-N-(3-methylbenzo[b]thiophen-2-ylmethyl)-3-(7-oxo-5,6,7,8-tetrahydro-1,8-naphthyridin-3-yl)acrylamide;
(E)-3-(6-Aminopyridin-3-yl)-N-methyl-N-(1-methyl-1H-pyrrolo[2,3-b]pyridin-3-ylmethyl)acrylamide;
(E)-3-(6-Aminopyridin-3-yl)-N-(1,7-dimethyl-1H-indol-3-ylmethyl)-N-methylacrylamide;
(E)-3-(6-Aminopyridin-3-yl)-N-(1,5-dimethyl-1H-indol-3-ylmethyl)-N-methylacrylamide;
(E)-N-Methyl-N-(2-methyl-1H-indol-3-ylmethyl)-3-(7-oxo-5,6,7,8-tetrahydro-1,8-naphthyridin-3-yl)acrylamide;
(E)-3-(6-Aminopyridin-3-yl)-N-(1,6-dimethyl-1H-indol-3-ylmethyl)-N-methylacrylamide;
(E)-3-(6-Aminopyridin-3-yl)-N-(2,3-dihydro-1H-3a-azacyclopenta[α]inden-8-yl)-N-methylacrylamide;
(E)-3-(6-Aminopyridin-3-yl)-N-methyl-(2-methylbenzo[b]thiophen-3-ylmethyl)acrylamide;
(E)-3-(6-Aminopyridin-3-yl)-N-(1-benzyl-1H-indol-3-ylmethyl)-N-methylacrylamide;
(E)-N-Methyl-N-(1-methyl-1H-indol-3-ylmethyl)-3-(3-oxo-3,4-dihydro-2H-pyrido[3,2-b]-1,4-oxazin-7-yl)acrylamide;or
(E)-N-Methyl-N-(1-methyl-1H-indol-2-ylmethyl)-3-(7-oxo-5,6,7,8-tetrahydro-1,8-naphthyridin-3-yl)propionamide;
or a pharmaceutically acceptable salt thereof.

Other compounds useful for treatment of bovine mastitis disclosed herein include:
(E)-N-methyl-N-((3-methylbenzofuran-2-yl)methyl)-3-(7-oxo-5,6,7,8-tetrahydro-1,8-naphthyridin-3-yl)acrylamide hydrochloride;
(E)-N-methyl-N-((3-methylbenzofuran-2-yl)methyl)-3-(7-oxo-5,6,7,8-tetrahydro-1,8-naphthyridin-3-yl)acrylamide hydrobromide;
(E)-N-methyl-N-((3-methylbenzofuran-2-yl)methyl)-3-(7-oxo-5,6,7,8-tetrahydro-1,8-naphthyridin-3-yl)acrylamide sulfate;
(E)-N-methyl-N-((3-methylbenzofuran-2-yl)methyl)-3-(7-oxo-5,6,7,8-tetrahydro-1,8-naphthyridin-3-yl)acrylamide methane sulfonate;
(E)-N-methyl-N-((3-methylbenzofuran-2-yl)methyl)-3-(7-oxo-5,6,7,8-tetrahydro-1,8-naphthyridin-3-yl)acrylamide ethane sulfonate;
(E)-N-methyl-N-((3-methylbenzofuran-2-yl)methyl)-3-(7-oxo-5,6,7,8-tetrahydro-1,8-naphthyridin-3-yl)acrylamide 2-hydroxyethanesulfonate;
(E)-N-methyl-N-((3-methylbenzofuran-2-yl)methyl)-3-(7-oxo-5,6,7,8-tetrahydro-1,8-naphthyridin-3-yl)acrylamide 4-methylbenzenesulfonate;
(E)-N-methyl-N-((3-methylbenzofuran-2-yl)methyl)-3-(7-oxo-5,6,7,8-tetrahydro-1,8-naphthyridin-3-yl)acrylamide 4-methylbenzenesulfonate monohydrate;
(E)-N-methyl-N-((3-methylbenzofuran-2-yl)methyl)-3-(7-oxo-5,6,7,8-tetrahydro-1,8-naphthyridin-3-yl)acrylamide benzenesulfonate;
(E)-N-methyl-N-((3-methylbenzofuran-2-yl)methyl)-3-(7-oxo-5,6,7,8-tetrahydro-1,8-naphthyridin-3-yl)acrylamide phosphate;
(E)-N-methyl-N-((3-methylbenzofuran-2-yl)methyl)-3-(7-oxo-5,6,7,8-tetrahydro-1,8-naphthyridin-3-yl)acrylamide trifluoroacetate;
(E)-N-methyl-N-((3-methylbenzofuran-2-yl)methyl)-3-(7-oxo-5,6,7,8-tetrahydro-1,8-naphthyridin-3-yl)acrylamide trichloroacetate;
(E)-6-(3-(methyl((3-methylbenzofuran-2-yl)methyl)amino)-3-oxoprop-1-enyl)-2-oxo-3,4-dihydro-1,8-naphthyridin-1(2H)-ylphosphonic acid;
Calcium (E)-6-(3-(methyl((3-methylbenzofuran-2-yl)methyl)amino)-3-oxoprop-1-enyl)-2-oxo-3,4-dihydro-1,8-naphthyridin-1(2H)-ylphosphonate;
Magnesium (E)-6-(3-(methyl((3-methylbenzofuran-2-yl)methyl)amino)-3-oxoprop-1-enyl)-2-oxo-3,4-dihydro-1,8-naphthyridin-1(2H)-ylphosphonate;
Disodium (E)-6-(3-(methyl((3-methylbenzofuran-2-yl)methyl)amino)-3-oxoprop-1-enyl)-2-oxo-3,4-dihydro-1,8-naphthyridin-1(2H)-ylphosphonate;
Dipotassium (E)-6-(3-(methyl((3-methylbenzofuran-2-yl)methyl)amino)-3-oxoprop-1-enyl)-2-oxo-3,4-dihydro-1,8-naphthyridin-1(2H)-ylphosphonate;
(Z)-N-methyl-N-((3-methylbenzofuran-2-yl)methyl)-3-(7-oxo-5,6,7,8-tetrahydro-1,8-naphthyridin-3-yl)acrylamide and rotamers thereof;
or a pharmaceutically acceptable salt thereof.

Compounds also contemplated include all specific compounds listed in the following U.S. patents and patent application publications: U.S. Pat. Nos. 6,503,903, by Miller et al., issued Jan. 7, 2003; 6,559,172, by Heerding et al., issued May 6, 2003; 6,573,272, by Miller et al., issued Jun. 3, 2003; 6,730,684, by Miller et al., issued May 4, 2004; 6,762,201, by Miller et al., issued Jul. 13, 2004; 6,846,819, by Miller et al., issued Jan. 25, 2005; 7,049,310, by Burgess et al., issued May 23, 2006; 7,790,709, by Berman et al., issued Sep. 7, 2010; and 7,879,872, by Berman et al., issued Feb. 1, 2011; and U.S. Pat. Application Publication Nos. 2009/0042927, by Pauls et al., filed Feb. 15, 2008; 2009/0156578, by Pauls et al., filed Dec. 1, 2006; 2010/0093705, by Sargent et al., filed Jun. 6, 2005; and 2010/0130470, by Pauls et al., filed Jul. 19, 2007, each of which is hereby incorporated by reference in their entirety.

Compositions disclosed herein may include a disclosed compound and an excipient, such as an excipient that is acceptable for veterinary use. For example, methods disclosed here may include administration of disclosed compositions topically (e.g. to udder and teats of a cow), subcutaneously, intravenously, and/or orally. Disclosed compositions can be used for various applications with the application route and dosage regimen are dictated by the frequency of milking and/or the skin condition of the animal. As an example of possible applications, contemplated compositions can be used in mammals as a pre- and post-milking application to decrease the potential for mastitis, and/or subcutaneous dermatological pathologies stemming from microbial infections, e.g. by administering disclosed compositions to mammalian skin, specifically the udder and teats of milking animals. Such compositions can be applied as a cleanser, scrub (cleanser with abrasive properties), lotion, or gel. For example, compositions can be used in both a cleanser or a scrub composition to help heal udder and teat skin which has been damaged by frequent milking. Additional applications for a contemplated sanitizer application within the disclosed methods include vaginal cleansers, calving sanitizers, burn disinfectants, wound healing aids, and perianal and colostomy wipe applications. For wipes, a contemplated formulation that includes a disclosed compound may be applied to paper or cloth towelettes, for use in administering the compound to a mammal for mastitis.

In some cases, mastitis may be transmitted, for example, through contact with surfaces contaminated with an infective organism (e.g., hands, equipment, etc.). In some cases, mastitis may be transmitted by contact with a milking machine. Thus, in some embodiments, an udder may be treated with disclosed compositions prior to, during, and/or after contact with a potentially contaminated surface. For example, a female mammal producing milk may be administered a contemplated compound by teat dipping (either post- pre-milking) or dry cow treatment to prevent or control mastistis.

In some embodiments, a contemplated method may include intramammary infusion of a disclosed compound or composition.

In some instances, depending on locale, products produced by an animal treated with a disclosed composition may not be marketed unless the composition and/or metabolites thereof have fallen below a threshold level in the animal. Accordingly, in some embodiments, an animal may be treated with a disclosed composition, for example, to treat or prevent mastitis and the treatment may be reduced or suspended for a period of time to allow levels of the composition to fall below the threshold level.

Disclosed compositions for use in the disclosed method may contain, for example, a disclosed compound and a surfactant or mixture thereof. Typically, a disclosed compound is present in a composition in a biologically effective, therapeutic, non-toxic concentration. Compositions may, in some embodiments, include a keratolytic agent or mixture and/or emollient or emollient system (e.g., water soluble refatting agent, glycerin, branched chain esters, ethoxylated partial glyceride fatty acid esters, protein derivatives, lanolin and lanolin derivatives, and fatty alcohol ethoxylates, emollient oils, fatty acids, and esters of fatty alcohols, or combinations thereof.) For example, a composition may include an effective amount of an emollient to condition the udder and teats of a cow for high frequency milking.

In some embodiments, disclosed methods further include administering an antibiotic such as pencillin, or other drug to the female mammal, such as further administration of oxytocin to stimulate milk let down.

In some embodiments, a disclosed composition effectively reduces susceptibility to mastitis (e.g., bovine mastitis) when used daily to treat the udder and teats of a mammal.

Also contemplated herein are methods of treating bovine E. coli infection, bovine Salmonella infection, bovine Mycoplasma infection, bovine S. aureus infection, bovine hemorrhagic septicemia, bovine contiguous pleuropneumonia, bovine mastitis, porcine E. coli infection, porcine Salmonella infection, porcine Pasteurella infection, porcine S. aureus infection, Aureporcine Mycoplasma infection, porcine atrophic rhinitis, porcine exudative epidermitis, avian E. coli infection, chicken pullorum, avian paratyphoid, avian cholera, avian infectious coryza, avian staphylococcus infection, avian Mycoplasma infection, canine E. coli septicemia, canine Salmonella infection, canine hemorrhagic septicemia, canine uterus empyema, canine cystitis, feline pleurisy, feline cystitis, feline Haemophilus infection, feline diarrhea, feline staphylococcus infection, and feline Mycoplasma infection.

In other embodiments, methods of treating or ameliorating osteomylitis, pneumonia, metritis, abscess, and/or wounds, in domesticated animals such as cows, goats, pigs, and small animal pets (e.g. cats or dogs).

U.S. Pat. Nos. 6,846,819; 7,049,310; 6,503,903; 6,673,941; 6,573,272; 6,762,201; 6,703,684; U.S. patent application Ser. Nos. 10/537,747; 11/628,569; 12/095977; 12/374,444 and 12/032001 are individually and specifically incorporated herein by reference as if set forth in full.

The embodiments described herein can be further understood by reference to the following non-limiting examples.

The examples and other embodiments described herein are exemplary and not intended to be limiting in describing the full scope of compositions and methods. Equivalent changes, modifications and variations of specific embodiments, materials, compositions and methods may be made within the scope of the present invention, with substantially similar results.

### EXAMPLES

### Preparation 1

Preparation of 2-methyl-3-(methylaminomethyl)indole

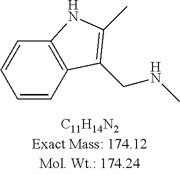

To a solution of 2-methylindole-3-carboxaldehyde (10.00 g. 62.84 mmole) in MeOH (100 mL) was added 2M CH₃NH₂ in MeOH (200 mL). After stirring for 3 hours at RT, the reaction solution was concentrated to a yellow oil which solidified under vacuum. This solid was dissolved in ethanol (350 mL) and NaBH₄ (2.38 g. 62.8 mmole) was added. The reaction was stirred at RT for 6 hours, then was concentrated under vacuum. The remaining residue was diluted with saturated aqueous Na₂CO₃ (50 mL) and extracted with EtOAc (2×200 mL). The organic phase was separated, washed with brine, and dried over Na₂SO₄. Flash chromatography on silica gel (9:1 CHCl₃/MeOH containing 5% NH₄OH) and drying under high vacuum gave the title compound (6.88 g, 63%) as a faintly yellow viscous solid: MS (ES) m/e 175 (M+H)⁺.

### Preparation 2

Preparation of 2-Amino-5-bromo-3-(hydroxymethyl)pyridine

a) 2-Amino-3-(hydroxymethyl)pyridine

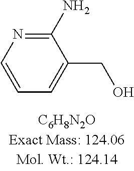

To a solution of 2-aminonicotinic acid (20.5 g, 148.1 mmole) in THF was added lithium aluminum hydride (300 mL, 1.0 M in THF) over 30 minutes. The reaction solution was heated to reflux for 18 hrs and then was cooled to room temperature. The reaction was quenched by the sequential dropwise addition of H₂O (11.5 mL), 15% NaOH (11.5 mL), and H₂O (34.5 mL). The mixture was stirred for 15 min, then was filtered through celite®, and the filter pad was washed thoroughly with THF followed by 5% CH₃OH/CHCl₃. The filtrate was concentrated to give the title compound (15.24 g, 83%) as a waxy light yellow solid: MS (ES) m/e 125 (M+H)⁺.

b) 2-Amino-5-bromo-3-(hydroxymethyl)pyridine

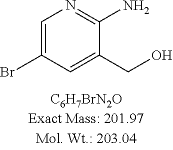

To a solution of 2-amino-3-(hydroxymethyl)pyridine (13.0 g, 116.0 mmole) in CH₂Cl₂ (300 mL) at RT was added NBS (22.71 g, 127.6 mmole). After stirring at RT for 45 min the reaction solution was concentrated and the residue was dissolved in CHCl₃. The resulting suspension was filtered and the filtrate was concentrated to a dark oil. Purification on silica gel (EtOAc) afforded the title compound (78%, 18.36 g) as a tan solid: MS (ES) m/e 204 (M+H)⁺.

### Preparation 3

Preparation of 6-bromo-3,4-dihydro-1H-1,8-naphthyridin-2-one

a) 2-Amino-5-bromo-3-(bromomethyl)pyridine hydrobromide

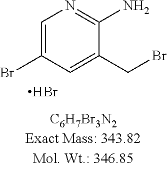

A solution of 2-amino-5-bromo-3-hydroxymethylpyridine (5.00 g. 24.6 mrnole), from Preparation 2, in 48% aqueous HBr (50 mL), was heated at reflux for 12 hrs. The reaction was concentrated and toluene was used to azeotrope the residual H₂O. The resulting light brown solid was placed under high vacuum overnight and used directly.

b) Methyl (±)-6-bromo-2-oxo-1,2,3,4-tetrahydro-1H-1,8-naphthyridine-3-carboxylate

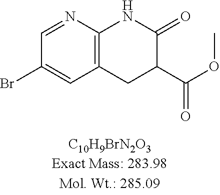 [SMILES: COC(=O)C1Cc2cc(Br)cnc2NC1=O]

To a solution of sodium methoxide (20.57 mL, 25% wt in CH₃OH) in CH₃OH (75 mL) was added dimethyl malonate (11.87 g, 89.9 mmole). After 30 min the 2-amino-5-bromo-3-(bromomethyl)pyridine hydrobromide salt prepared above was added to the methoxide solution and the reaction was stirred at RT overnight. The reaction slurry was concentrated to dryness under vacuum and then suspended in 1:1 H₂O/Et₂O. The remaining solids were filtered and washed with H₂O then with hexanes to afford the title compound (4.08 g, 58%) as a white solid after drying: MS (ES) m/e 286 (M+H)^{±}.

c) 6-Bromo-3,4-dihydro-1H-1,8-naphthyridin-2-one

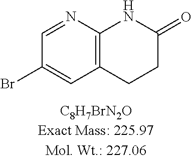 [SMILES: O=C1CCc2cc(Br)cnc2N1]

To a solution of methyl (±)-6-bromo-2-oxo-1,2,3,4-tetrahydro-1H-1,8-naphthyridine-3-carboxylate (2.00 g, 7.0 mmole) in CH₃OH (75 mL) was added 1.0 M NaOH (30 mL). The reaction was heated to reflux for 4 hrs and then cooled to RT. The reaction was neutralized with 1.0 M HCl (30 mL) then was heated at reflux overnight. The reaction slurry was concentrated to dryness and the residues was suspended in 95:5 CHCl₃/CH₃OH. The solids were removed by filtration and the filtrate was concentrated to afford the title compound (1.40 g, 88%) as an off-white solid: MS (ES) m/e 228 (M+H)^{±}.

### Example 1

Preparation of (E)-N-methyl-N-(2-methyl-1H-indol-3-ylmethyl)-3-(7-oxo-5,6,7,8-tetrahydro-1,8-naphthyridin-3-yflacrylamide

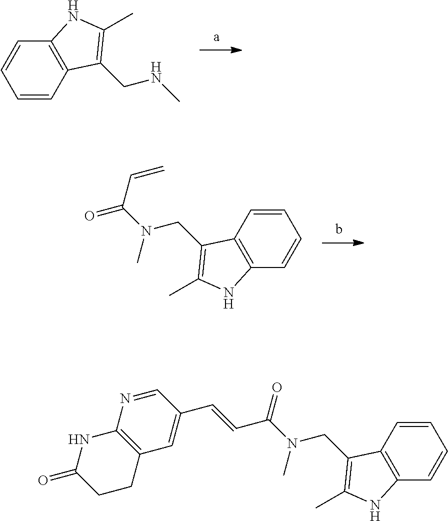 [SMILES: CNCc1c(C)[nH]c2ccccc12 ; C=CC(=O)N(C)Cc1c(C)[nH]c2ccccc12 ; Cc1[nH]c2ccccc2c1CN(C)C(=O)/C=C/c1cnc2c(c1)CCC(=O)N2]

(a) acryloyl chloride, Et₃N, CH₂Cl₂; (b) 6-bromo-3,4-dihydro-1H-1,8-naphthyridin-2-one, Pd(OAc)₂, P(o-tol)₃, (i-Pr)₂EtN, EtCN

a) N-methyl-N-((2-methyl-1H-indol-3-yl)methyl)acrylamide

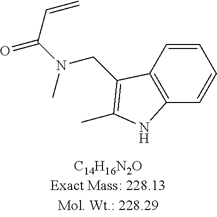 [SMILES: C=CC(=O)N(C)Cc1c(C)[nH]c2ccccc12]

To a solution of 2-methyl-3-(methylaminomethyl)indole (1.40 g, 8.00 mmole), from Preparation 1, and triethylamine in CH₂Cl₂ at 0° C. was added a solution of acryloyl chloride in CH₂Cl₂. The reaction was stirred at 0° C. for 1 hr and then poured into water. The layers were separated, and the organic layer was washed with brine, dried over MgSO₄, and concentrated in vacuo to yield the title compound.

b) (E)-N-methyl-N-(2-methyl-1H-indol-3-ylmethyl)-3-(7-oxo-5,6,7,8-tetrahydro-1,8-naphthyridin-3-yl)acrylamide

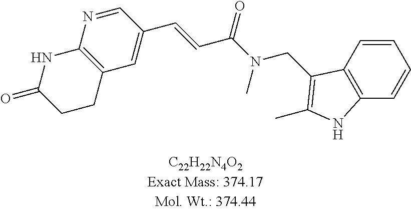 [SMILES: Cc1[nH]c2ccccc2c1CN(C)C(=O)/C=C/c1cnc2c(c1)CCC(=O)N2]

A mixture of 6-bromo-3,4-dihydro-1H-1,8-naphthyridin-2-one, from Preparation 3, and N-methyl-N-((2-methyl-1H-indol-3-yl)methyl)acrylamide in propionitrile was treated with (i-Pr)₂EtN, palladium acetate, and (o-tolyl)₃P, and the resulting mixture was heated at gentle reflux. After 18 hr, the reaction was cooled, filtered through Celite®, and concentrated. Flash chromatography on silica gel (2% MeOH/CH₂Cl₂ gave the tide compound (1.30 g, 65%) as a light yellow solid: MS (ES) m/e 376 (M+H)⁺.

### Example 2

Preparation of (E)-N-Methyl-N-(3-methylbenzofuran-2-ylmethyl)-3-(2,3,4,5-tetrahydro-1H-pyrido[2,3-e][1,4]diazepin-7-yl)acrylamide hydrochloride

i) Preparation of (E)-7-{2-[Methyl-(3-methylbenzofuran-2-ylmethyl)carbamoyl]vinyl}-1,2,3,5-tetrahydropyrido [2,3-e][1,4]diazepine-4-carboxylic acid tert-butyl ester

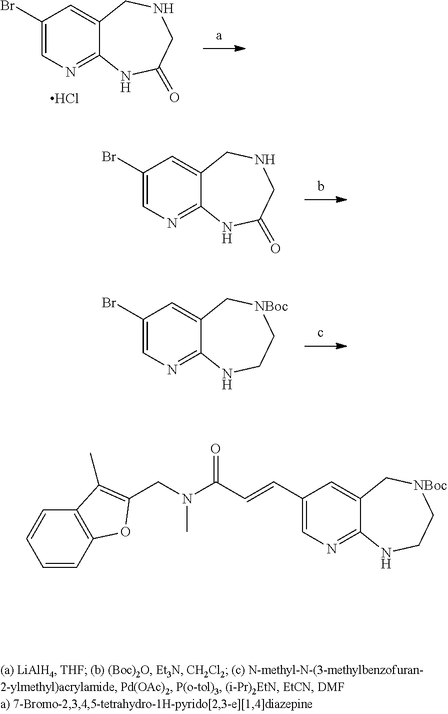 [SMILES: O=C1CNCc2cc(Br)cnc2N1 ; Cl ; O=C1CNCc2cc(Br)cnc2N1 ; CC(C)(C)OC(=O)N1CCNc2ncc(Br)cc2C1 ; Cc1c(CN(C)C(=O)/C=C/c2cnc3c(c2)CN(C(=O)OC(C)(C)C)CCN3)oc2ccccc12]

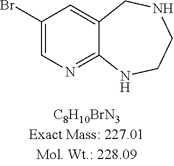 [SMILES: Brc1cnc2c(c1)CNCCN2]

A suspension of 7-bromo-1,3,4,5-tetrahydro-pyrido[2,3-e][1,4]diazepin-2-one hydrochloride (1.16 g, 4.16 mmol) in THF (35 mL) was cooled in an ice bath and treated dropwise with LiAlH₄ (8.4 mL of a 1.0 M solution in THF, 8.4 mmol). After stirring for 30 min, the ice bath was removed and the solution was allowed to warm to room temperature.

After heating to reflux overnight, the mixture was cooled in an ice bath. The reaction was quenched sequentially with H₂O (0.3 mL), 15% NaOH (0.3 mL) and H₂O (0.9 mL). After 5 min, the ice bath was removed and the mixture was stirred at room temperature for 2.5 h. The mixture was filtered through Celite®, and the filtrate was concentrated in vacuo to give a yellow syrup. Purification by flash column chromatography (silica gel, CH₂Cl₂/MeOH, 95:5 to 90:10) gave the title compound (0.42 g, 44%) as a white solid: ¹H NMR (300 MHz, CDCl₃) 8 8.03 (d, J=2.3 Hz, 1H), 7.44 (d, J=2.0 Hz, 1H), 4.96 (br s, 1H), 3.82 (s, 2H), 3.22-3.15 (m, 2H), 3.08-3.05 (m, 2H), 1.97 (br s, 1H); MS (ESI) m/e 228 (M+H)⁺.

b) 7-Bromo-1,2,3,5-tetrahydro-pyrido[2,3-e][1,4]diazepine-4-carboxylic acid tert-butyl ester

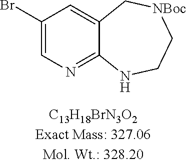 [SMILES: CC(C)(C)OC(=O)N1CCNc2ncc(Br)cc2C1]

A solution of 7-bromo-2,3,4,5-tetrahydro-1H-pyrido[2,3-e][1,4]diazepine (0.42 g, 1.8 mmol) in CH₂Cl₂ (20 mL) was treated with Et₃N (0.34 mL, 2.4 mmol) followed by di-tert-butyl-dicarbonate (0.44 g, 2.0 mmol). After stirring for 1 h, the reaction was concentrated to a white solid. Purification by flash column chromatography (silica gel, CH₂Cl₂/MeOH, 99:1) gave the title compound (0.55 g, 91%) as a white solid and as a mixture of rotamers: ¹H NMR (300 MHz, CDCl₃) δ 8.06 (s, 1H), 8.59-8.45 (m, 1H), 4.90 (s, 1H), 4.35-4.27 (m, 2H), 3.66-3.65 (m, 2H), 3.29-3.24 (m, 2H), 1.42 (s, 9H); MS (ESI) m/e 328 (M+H)⁺.

iii) (E)-7-{2-[Methyl-(3-methylbenzofuran-2-ylmethyl)carbamoyl]vinyl}-1,2,3,5-tetrahydro-pyrido[2,3-e][1,4]diazepine-4-carboxylic acid tert-butyl ester

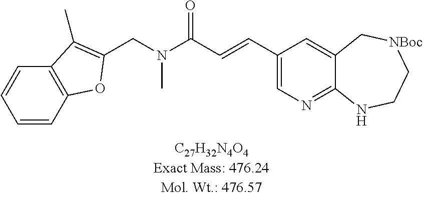 [SMILES: Cc1c(CN(C)C(=O)/C=C/c2cnc3c(c2)CN(C(=O)OC(C)(C)C)CCN3)oc2ccccc12]

A solution of 7-bromo-1,2,3,5-tetrahydro-pyrido[2,3-e][1,4]diazepine-4-carboxylic acid tert-butyl ester (0.53 g, 1.6 mmol) and N-methyl-N-(3-methylbenzofuran-2-ylmethyl)acrylamide (0.41 g, 1.8 mmol) in propionitrile (8.0 mL) and DMF (2.0 mL) was de-oxygenated with Ar for 10 min. The mixture was treated with (i-Pr)₂EtN (0.62 mL, 3.5 mmol) and was de-oxygenated with Ar for 5 min. Pd(OAc)₂ (36 mg, 0.16 mmol) and P(o-tol)₃ (100 mg, 0.33 mmol) were added simultaneously, and the mixture was de-oxygenated a third time for 10 min. The mixture was heated to reflux for 6 h, then allowed to cool. The mixture was diluted with EtOAc (100 mL) and washed with H₂O (50 mL). The organic layer was dried over Na₂SO₄, filtered and concentrated to an orange oil. Purification by flash column chromatography (silica gel, CH₂Cl₂/MeOH, 98:2) gave the title compound (0.48 g, 62%) as a white powder and as a mixture of amide rotamers: ¹H NMR (300 MHz, DMSO-d₆) δ 8.15-8.10 (m, 1H), 7.87-7.74 (m, 1H), 7.57-7.42 (m, 3H), 7.32-6.77 (m, 4H), 4.97-4.78 (m, 2H), 4.51-4.42 (m, 2H), 3.59-3.57 (m, 2H), 3.43-3.41 (m, 2H), 3.17-2.92 (m, 3H), 2.26 (s, 3H), 1.38-1.24 (m, 9H); MS (ESI) m/e 477 (M+H)⁺.

ii) Preparation of (E)-N-Methyl-N-(3-methylbenzofuran-2-ylmethyl)-3-(2,3,4,5-tetrahydro-1H-pyrido[2,3-e][1,4]diazepin-7-yl)acrylamide hydrochloride

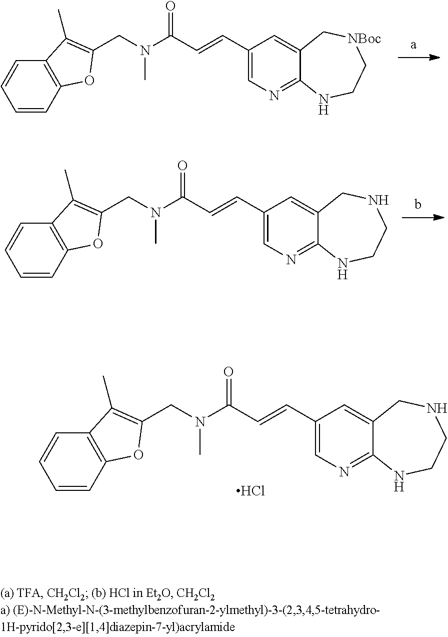 [SMILES: Cc1c(CN(C)C(=O)/C=C/c2cnc3c(c2)CN(C(=O)OC(C)(C)C)CCN3)oc2ccccc12 ; Cc1c(CN(C)C(=O)/C=C/c2cnc3c(c2)CNCCN3)oc2ccccc12 ; Cc1c(CN(C)C(=O)/C=C/c2cnc3c(c2)CNCCN3)oc2ccccc12 ; Cl]

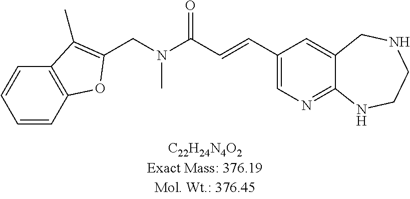 [SMILES: Cc1c(CN(C)C(=O)/C=C/c2cnc3c(c2)CNCCN3)oc2ccccc12]

A solution of (E)-7-{2-[methyl-(3-methylbenzofuran-2-ylmethyl)carbamoyl]vinyl}-1,2,3,5-tetrahydro-pyrido[2,3-e][1,4]diazepine-4-carboxylic acid tert-butyl ester (0.38 g, 0.80 mmol) in CH₂Cl₂ (4 mL) was cooled in an ice bath and then treated with TFA (4 mL). After stirring for 2 h, the mixture was concentrated under vacuum. The residue was treated with saturated NaHCO₃ (25 mL) and extracted with CH₂Cl₂/MeOH (4×50 mL of a 98:2 mixture). The combined organic layers were dried over Na₂SO₄, filtered and concentrated to a light yellow solid. Purification by flash column chromatography (silica gel, CH₂Cl₂/MeOH, 92:8) gave the title compound (0.21 g, 70%) as a white powder and as a mixture of amide rotamers: ¹H NMR (300 MHz, DMSO-d₆) δ 8.14 (br s, 1H), 7.68-7.63 (m, 1H), 7.50-7.40 (m, 3H), 7.26-7.20 (m, 2H), 7.04-6.72 (m, 1H), 5.10 (s, 1H), 4.83-4.72 (m, 2H), 3.89 (s, 2H), 3.30-3.26 (m, 2H), 3.22-3.04 (m, 5H), 2.31 (s, 3H), 1.70 (br s, 1H); MS (ESI) m/e 377 (M+H)⁺.

b) (E)-N-Methyl-N-(3-methylbenzofuran-2-ylmethyl)-3-(2,3,4,5-tetrahydro-1H-pyrido[2,3-e][1,4]diazepin-7-yl)acrylamide hydrochloride

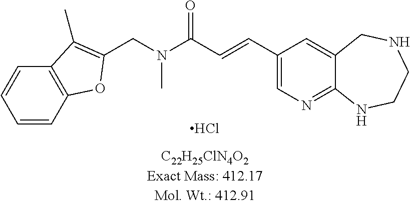 [SMILES: Cc1c(CN(C)C(=O)/C=C/c2cnc3c(c2)CNCCN3)oc2ccccc12 ; Cl]

A solution of (E)-N-methyl-N-(3-methylbenzofuran-2-ylmethyl)-3-(2,3,4,5-tetrahydro-1H-pyrido[2,3-e][1,4]diazepin-7-yl)acrylamide (0.21 g, 0.56 mmol) in CH₂Cl₂ (5 mL) was treated with anhydrous HCl (0.56 mL of a 1.0 M solution in Et₂O, 0.56 mmol). After stirring for 5 min, the mixture was diluted with Et₂O (50 mL), allowed to stir for 30 min and sonicated for 5 min. The solid was isolated by filtration, washed with Et₂O, and dried under vacuum at 50° C. for 4 days to give the title compound (0.22 g, 97%) as an off-white powder and as a mixture of amide rotamers: ¹H NMR (300 MHz, DMSO-d₆) δ 9.66 (br s, 2H), 8.36-8.33 (m, 1H), 8.14 (s, 1H), 7.58-7.07 (m, 7H), 4.98-4.79 (m, 2H), 4.26 (s, 2H), 3.51 (s, 2H), 3.33 (s, 2H), 3.17-2.91 (m, 3H), 2.27 (s, 3H); MS (ESI) m/e 377 (M+H)⁺.

### Example 3

Preparation of (E)-N-methyl-N-(3-methylbenzofuran-2-ylmethyl)-3-(7-oxo-5,6,7,8-tetrahydro-1,8-naphthyridin-3-yl)acrylamide

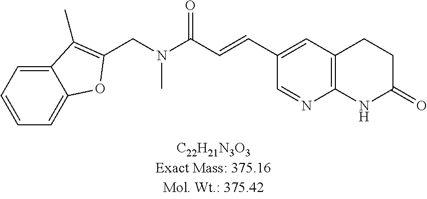 [SMILES: Cc1c(CN(C)C(=O)/C=C/c2cnc3c(c2)CCC(=O)N3)oc2ccccc12]

The title compound is prepared following methods analogous to those described in the previous preparations and examples and as described in U.S. Pat. No. 7,049,310, by Burgess et al., issued on May 23, 2006, which is incorporated herein by reference.

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments have been discussed, the above specification is illustrative and not restrictive. Many variations will become apparent to those skilled in the art upon review of this specification. The full scope of the embodiments should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

What is claimed is:

1. A method of treating mastitis in a female mammal in need thereof, comprising administering to the female mammal having or at risk of having mastitis an effective amount of a compound selected from the group consisting of (E)-N-methyl-N-((2-methyl-1H-indol-3-yl)methyl)-3-(7-oxo-5,6,7,8-tetrahydro-1,8-naphthyridin-3-yl)acrylamide; (E)-N-methyl-N-((3-methylbenzofuran-2-yl)methyl)-3-(2,3,4,5-tetrahydro-1H-pyrido[2,3-e][1,4]diazepin-7-yl)acrylamide; (E)-N-methyl-N-(3-methylbenzofuran-2-ylmethyl)-3-(7-oxo-5,6,7,8-tetrahydro-1,8-naphthyridin-3-yl)acrylamide; and pharmaceutically acceptable salts and esters thereof

2. The method of claim 1, wherein the female mammal is a milk producing mammal.

3. The method of claim 1, wherein the female mammal is a cow, horse, human, goat, sheep, buffalo, or camel.

4. A method of treating bovine mastitis in a cow in need thereof, comprising administering to said cow an effective amount of a composition comprising (E)-N-methyl-N-((2-methyl-1H-indol-3-yl)methyl)-3-(7-oxo-5,6,7,8-tetrahydro-1,8-naphthyridin-3-yl)acrylamide or a pharmaceutically acceptable salt or ester thereof

5. A method of treating bovine mastitis in a cow in need thereof, comprising administering to said cow an effective amount of a composition comprising (E)-N-methyl-N-((3-methylbenzofuran-2-yl)methyl)-3-(2,3,4,5-tetrahydro-1H-pyrido[2,3-e][1,4]diazepin-7-yl)acrylamide or a pharmaceutically acceptable salt or ester thereof.

6. The method of claim 1, wherein the mastitis is caused by a bacterial infection.

7. The method of claim 6, wherein the bacterial infection is caused by one or more strains of Staphylococcus aureus.

8. The method of claim 7, wherein the bacterial infection is caused by one or more strains of Staphylcoccus Oxford, Staphylococcus aureus WCUH29, Streptococcus pneumoniae ERY2, Streptococcus pneumoniae 1629, Streptococcus pneumoniae N 1387, Enterococcus faecalis I, Enterococcus faecalis 7, Haemophilus influenzae Q1, Haemophilus influenzae NEMC1, Moraxella Catarrhalis 1502, Escherichia coli 7623 AcrABEFD+, Escherichia coli 120 AcrAB-, Escherichia coli MG1655, or Escherichia coli MG1658.

9. The method of claim 6, wherein the bacterial infection is caused by one or more strains of Staphylococcus auereues, Streptococcus dysgalactiae, Streptococcus equinus, Streptococcus agalactiae, Staphylococcus hyicus, Staphylococcus simulans, Staphylococcus epidermidis, Staphylococcus chromogenes or Staphylococcus xylosus.

10. The method of claim 6, wherein the bacterial infection is caused by one or more strains of Pseudomonas aeruginosa, Corynebacterium pyogenes, Mycoplasma bovis, Serratia, Candida, E. coli, Klebsiella or Enterobacter.

11. The method of claim 6, wherein the S. aureus is methicillin-resistant Staphylococcus aureus.

12. The method of claim 3, wherein the compound is administered to the udder of the cow.

13. The method of claim 1, wherein the compound is administered orally, rectally, vaginally, subcutaneously, or intravenously.
